SEQUENCE LISTING (37 sequences)

SEQ ID NO: 1 - protein, 328 aa, synthetic construct
MYRMQLLSCIALSLALVTNSDIQMTQSPSSLSASVGDRVTITCQASQSPNNLLAWYQQKPGKAPKLLIYGASDLPSGVPSRFSGSGSGTDFTLTISSLOPEDFATYYCONNYYVGPVSYAFGGGTKVEIKGGGGSGGGGSGGGGSQVQLVQSGAEVKKPGASVKVSCKVSGYSLSKYDMSWVRQAPGKGLEWMGIIYTSGYTDYAQKFOGRVTMTEDTSTDTAYMELSSLRSEDTAVYYCATGNPYYTNGFNSWGQGTLVTVSSTTTPAPRPPTPAPTIASQPLSLRPEACRPAAAVHTRGLDFACDAAVLVLLVIVIISLIVLVVIW

SEQ ID NO: 2 - protein, 20 aa, Homo sapiens
MYRMQLLSCIALSLALVTNS

SEQ ID NO: 3 - protein, 43 aa, Homo sapiens
TTTPAPRPPTPAPTIASQPLSLRPEACRPAAAVHTRGLDFACD

SEQ ID NO: 4 - protein, 21 aa, Homo sapiens
AAVLVLLVIVIISLIVLVVIW

SEQ ID NO: 5 - protein, 148 aa, Homo sapiens
NILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYM

SEQ ID NO: 6 - protein, 21 aa, Homo sapiens
VLFYLAVGIMFLVNTVLWVTI

SEQ ID NO: 7 - protein, 34 aa, Homo sapiens
KFVASNQKTIQPPRKAVEEPLNAFKESKGMMNDE

SEQ ID NO: 8 - protein, 508 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 9 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 10 - protein, 123 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE

SEQ ID NO: 11 - protein, 116 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVV

SEQ ID NO: 12 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 13 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 14 - protein, 178 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGD

SEQ ID NO: 15 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 16 - protein, 183 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKGQMEPRTHPTWLLHIFIPFCIIAFIFIATVIALRKQLCQKLYSSKDTTKRPVTTTKREVNSAI

SEQ ID NO: 17 - protein, 182 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKDGTQDPSNLAASHFHPLLHHCFHFHSHSDSPKKTTLSKAVFFKRHNKKTCHHNKEGSEQCYL

SEQ ID NO: 18 - protein, 13 aa, synthetic construct
KVSGYSLSKYDMS

SEQ ID NO: 19 - protein, 16 aa, synthetic construct
IIYTSGYTDYAQKFQG

SEQ ID NO: 20 - protein, 13 aa, synthetic construct
ATGNPYYTNGFNS

SEQ ID NO: 21 - protein, 11 aa, synthetic construct
QASQSPNNLLA

SEQ ID NO: 22 - protein, 8 aa, synthetic construct
YGASDLPS

SEQ ID NO: 23 - protein, 12 aa, synthetic construct
QNNYYVGPVSYA

SEQ ID NO: 24 - protein, 339 aa, synthetic construct
MALPVTALLLPLALLLHAARPEQKLISEEDLDIQMTQSPSSLSASVGDRVTITCQASQSPNNLLAWYQQKPGKAPKLLIYGASDLPSGVPSRFSGSGSGTDFTLTISSLOPEDFATYYCONNYYVGPVSYAFGGGTKVEIKGGGGSGGGGSGGGGSQVQLVQSGAEVKKPGASVKVSCKVSGYSLSKYDMSWVRQAPGKGLEWMGIIYTSGYTDYAQKFOGRVTMTEDTSTDTAYMELSSLRSEDTAVYYCATGNPYYTNGFNSWGQGTLVTVSSTTTPAPRPPTPAPTIASQPLSLRPEACRPAAAVHTRGLDFACDAAVLVLLVIVIISLIVLVVIW

SEQ ID NO: 25 - protein, 21 aa, Homo sapiens
MALPVTALLLPLALLLHAARP

SEQ ID NO: 26 - protein, 10 aa, synthetic construct
EQKLISEEDL

SEQ ID NO: 27 - protein, 339 aa, synthetic construct
MALPVTALLLPLALLLHAARPEQKLISEEDLQMQLVQSGGEVKKPGASVKVSCKTSGYRFTSYGISWVRQAPGQGLEWMGWISGYNGETNYAETLQGRLTLTTDTSTSTAYMELGSLRPDDTAVYYCTRDGHSPYFDYWGQGTLVTVSSGGGGSGGGGSGGGGSQAVLTQPASVSGSPGQSVTISCTGTSSDVGGYNYVSWYQQHPGKAPKLMIYEVSKRPSGIPERFSGSNSGNTATLTISRVEAGDEADYYCQVWDSSSDHWVFGRGTKLTVLTTTPAPRPPTPAPTIASQPLSLRPEACRPAAAVHTRGLDFACDAAVLVLLVIVIISLIVLVVIW

SEQ ID NO: 28 - protein, 21 aa, Homo sapiens
MALPVTALLLPLALLLHAARP

SEQ ID NO: 29 - protein, 118 aa, synthetic construct
QMQLVQSGGEVKKPGASVKVSCKTSGYRFTSYGISWVRQAPGQGLEWMGWISGYNGETNYAETLQGRLTLTTDTSTSTAYMELGSLRPDDTAVYYCTRDGHSPYFDYWGQGTLVTVSS

SEQ ID NO: 30 - protein, 15 aa, synthetic construct
GGGGSGGGGSGGGGS

SEQ ID NO: 31 - protein, 111 aa, synthetic construct
QAVLTQPASVSGSPGQSVTISCTGTSSDVGGYNYVSWYQQHPGKAPKLMIYEVSKRPSGIPERFSGSNSGNTATLTISRVEAGDEADYYCQVWDSSSDHWVFGRGTKLTVL

SEQ ID NO: 32 - protein, 350 aa, synthetic construct
MALPVTALLLPLALLLHAARPEQKLISEEDLEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARRKKRERGFSGNDPVGAIDVWGQGTLVTVSSGGGGSGGGGSGGGGSQSVLTQPPSASGTPGQRVTISCTGSSSNIGAGYDVHWYQQLPGTAPKLLIYGNTNRPSGVPDRFSGSKSGTSASLAISGLRSEDEADYYCSAWDDSLSGVVFGGGTKLTVLGTTTPAPRPPTPAPTIASQPLSLRPEACRPAAAVHTRGLDFACDAAVLVLLVIVIISLIVLVVIW

SEQ ID NO: 33 - protein, 128 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARRKKRERGFSGNDPVGAIDVWGQGTLVTVSS

SEQ ID NO: 34 - protein, 112 aa, synthetic construct
QSVLTQPPSASGTPGQRVTISCTGSSSNIGAGYDVHWYQQLPGTAPKLLIYGNTNRPSGVPDRFSGSKSGTSASLAISGLRSEDEADYYCSAWDDSLSGVVFGGGTKLTVLG

SEQ ID NO: 35 - protein, 335 aa, synthetic construct
MALPVTALLLPLALLLHAARPEQKLISEEDLEQSLEESGGGLVKPGASLTVTCAVSGFSLNSYAITWVRQAPGKGLEYIGYIDTGASITDYASWAKGRFTISKTSSTTVTLEMTSLTDADTATYFCARGFSMFKLWGPGTLVTISSGGGGSGGGGSGGGGSELDLTQTPSSVSAAVGGTVTISCQASQSVYGNNRLAWYHQKPGQPPKRLIYLASTLDSGVPSRFKGAGSGTQFTLTISDLECDDAATYYCAGGYAGNFNAFGGGTEVEIKTTTPAPRPPTPAPTIASQPLSLRPEACRPAAAVHTRGLDFACDAAVLVLLVIVIISLIVLVVIW

SEQ ID NO: 36 - protein, 115 aa, synthetic construct
EQSLEESGGGLVKPGASLTVTCAVSGFSLNSYAITWVRQAPGKGLEYIGYIDTGASITDYASWAKGRFTISKTSSTTVTLEMTSLTDADTATYFCARGFSMFKLWGPGTLVTISS

SEQ ID NO: 37 - protein, 110 aa, synthetic construct
ELDLTQTPSSVSAAVGGTVTISCQASQSVYGNNRLAWYHQKPGQPPKRLIYLASTLDSGVPSRFKGAGSGTQFTLTISDLECDDAATYYCAGGYAGNFNAFGGGTEVEIK

(19) United States
(12) Patent Application Publication
(10) Pub. No.: US 2025/0268949 A1
(43) Pub. Date: Aug. 28, 2025

(54) IMMUNE CELL INHIBITION BY IMMUNE CHECKPOINT ENGAGERS

(71) Applicant: THE REGENTS OF THE UNIVERSITY OF CALIFORNIA, Oakland, CA (US)

(72) Inventor: Tobias Deuse, Burlingame, CA (US)

(73) Assignee: THE REGENTS OF THE UNIVERSITY OF CALIFORNIA, Oakland, CA (US)

(21) Appl. No.: 18/853,080

(22) PCT Filed: Apr. 11, 2023
(86) PCT No.: PCT/US2023/018189

§ 371 Date: Sep. 30, 2024

Related U.S. Application Data
(60) Provisional application No. 63/330,539, filed on Apr. 13, 2022.
(60) Provisional application No. 63/448,081, filed on Feb. 24, 2023.

(51) Int. Cl. A61K 35/545; A61K 40/10; A61K 40/33; A61K 40/36; A61K 40/42; A61P 37/06; C07K 14/55; C07K 14/705; C07K 14/71; C07K 16/28

(52) U.S. Cl. CPC A61K 35/545; A61K 40/10; A61K 40/33; A61K 40/36; A61K 40/421; A61P 37/06; C07K 14/55; C07K 14/70517; C07K 14/71; C07K 16/2818; C07K 2317/55; C07K 2317/565; C07K 2317/622; C07K 2317/75; C07K 2317/92; C07K 2319/02

(57) ABSTRACT

The invention provides cells that have an increased immune checkpoint engagement function (Immune Checkpoint Engager “ICE” cells). The ICE cells comprise an engager molecule' expressed on a cell surface, wherein said engager molecule engages with an immune checkpoint molecule on an immune cell, wherein said engager molecule is expressed at least at a level that protects said ICE cell from being killed by said immune cell.

### I. CROSS REFERENCE TO RELATED APPLICATIONS

This application claims priority under 35 U.S.C. § 119(e) to U.S. Provisional Application No. 63/330,539, filed Apr. 13, 2022, and U.S. Provisional Application No. 63/448,081, filed Feb. 24, 2023 and are incorporated herein by reference in their entirety.

### II. FIELD OF THE INVENTION

The invention provides cells that have an increased immune checkpoint engagement (ICE) function. In some embodiments the cells have a Programmed Cell Death Protein 1 (PD-1) engagement function (PD-1 engager cells) that resist immune responses when transplanted into a subject when compared to a parental cell having an unmodified PD-1 engagement function. In some embodiments, the immune checkpoint engager cells are hypoimmune cells. In other embodiments, the cells are pluripotent cells. In other embodiments, the cells are embryonic stem (ES) cells or induced pluripotent stem cells (iPSC). In other embodiments, the immune checkpoint engager cells are differentiated somatic cells. In other embodiments, the immune checkpoint engager cells are hypoimmune pluripotent cells (HIP cells). In further embodiments, the HIP cells are blood type O (HIPO), Rhesus factor (Rh) negative (HIP−) or both type O and Rh−(HIPO−). In other embodiments, the immune checkpoint engager cells have been derived or differentiated from HIP, HIP−, or HIPO− cells. In other embodiments, the immune checkpoint engager cells comprise an antibody Fc receptor to protect against antibody dependent cellular cytotoxicity (ADCC) or complement dependent cytotoxicity (CDC). In other embodiments, the ICE cells express a ligand that binds to an inhibitory immune cell receptor selected from the group consisting of an LILRB1 engager, an LILRB3 engager, and a TIM3 engager. In other embodiments, the ICE cells are PD-1 engager cells that additionally express a ligand that binds to another inhibitory immune cell receptor selected from the group consisting of CD47, truncated CD47, a SIRPα engager, an LILRB1 engager, an LILRB3 engager, and a TIM3 engager.

### III. BACKGROUND OF THE INVENTION

Natural killer cells, or NK cells, are cytotoxic lymphocytes critical to the innate immune system. The role NK cells play is analogous to that of cytotoxic T cells in the vertebrate adaptive immune response. NK cells provide rapid responses to virus-infected and cancerous cells. Typically, NK cells become activated by target cells downregulating major histocompatibility complex (MHC) as this is one major inhibitory NK cell signal. NK cell activation triggers cytokine release resulting in lysis or apoptosis. NK cells are unique, because they can recognize stressed cells as they upregulate other stimulatory NK cell signals and do not require prior exposure to certain cell epitopes. This makes them very fast responders. They can also quickly respond to antibody-laden cells because binding of free antibody Fc is a strong stimulatory NK cell signal. NK cells do not require major activation to kill cells that are missing “self” markers of MHC class I other than some cytokine exposure like IL-2 or IL-15. This role is especially important because harmful cells that have downregulated or missing MHC I markers cannot be detected and destroyed by other immune cells such as T lymphocyte cells.

NK cells are large granular lymphocytes that are differentiated from the common lymphoid progenitor-generating B and T lymphocytes. They differentiate and mature in the bone marrow, lymph nodes, spleen, tonsils, and thymus, where they then enter into the circulation.

Programmed cell death protein 1, also known as PD-1 and CD279 (cluster of differentiation 279), is a protein on the surface of T, B, and some NK cells that has a role in regulating the immune system's response to the cells of the human body by down-regulating the immune system and promoting self-tolerance by suppressing T cell inflammatory activity. This prevents autoimmune diseases, but it can also prevent the immune system from killing cancer cells.

PD-1 often shows high and sustained expression levels during persistent antigen encounter, which can occur in the setting of chronic infections and cancer. PD-1 is an immune checkpoint that guards against autoimmunity through two mechanisms. First, it promotes apoptosis (programmed cell death) of antigen-specific T-cells in lymph nodes. Second, it reduces apoptosis in regulatory T cells (anti-inflammatory, suppressive T cells).

The PD-1/PD-L1 (ligand of PD-1) based pathway is of great value in immunotherapy of cancer and has become an important immune checkpoint. Antibody inhibitors of PD-1/PD-L1 interaction have shown clinical efficacy in many tumors.

The PD-1 protein in humans is encoded by the PDCD1 gene. PD-1 is a cell surface receptor that belongs to the immunoglobulin superfamily and is expressed on T cells and pro-B cells. PD-1 binds two ligands, PD-L1 and PD-L2.

Programmed death-ligand 1 (PD-L1) also known as cluster of differentiation 274 (CD274) or B7 homolog 1 (B7-H1) is a protein that in humans is encoded by the CD274 gene. PD-L1 is a 40 kDa type 1 transmembrane protein that has been speculated to play a major role in suppressing the adaptive arm of immune systems during particular events such as pregnancy, tissue allografts, autoimmune disease and other disease states such as hepatitis. Normally the adaptive immune system reacts to antigens that are associated with immune system activation by exogenous or endogenous danger signals. In turn, clonal expansion of antigen-specific CD8+ T cells and/or CD4+ helper cells is propagated. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM). This reduces the proliferation of antigen-specific T-cells in lymph nodes while simultaneously reducing apoptosis in regulatory T cells (anti-inflammatory, suppressive T cells). This is further mediated by a lower regulation of the gene Bcl-2.

Programmed cell death 1 ligand 2 (also known as PD-L2, B7-DC) is a protein that in humans is encoded by the PDCDILG2 gene. PDCDILG2 has also been designated as CD273 (cluster of differentiation 273). PDCDILG2 is an immune checkpoint receptor ligand which plays a role in negative regulation of the adaptive immune response. PD-L2 is one of two known ligands for Programmed cell death protein 1 (PD-1).

SIRPα is a member of the signal-regulatory-protein (SIRP) family and also belongs to the immunoglobulin superfamily. SIRP family members are receptor-type transmembrane glycoproteins known to be involved in the negative regulation of receptor tyrosine kinase-coupled signaling processes. SIRPα can be phosphorylated by tyrosine kinases. The phosphotyrosine residues recruit SH2 domain-containing tyrosine phosphatases (PTP) and serve as their substrates. SIRPα participates in signal transduction mediated by various growth factor receptors.

CD47 is a ligand for SIRPα. CD47 is a “marker-of-self” protein that can be overexpressed broadly across tumor types. It is emerging as a novel potent macrophage immune checkpoint for cancer immunotherapy. CD47 in tumor cells sends a “don't-eat-me” signal that inhibits macrophage phagocytosis. This presents opportunities and challenges for CD47 inhibitors both as a monotherapy and in combination treatments for hematological cancers and solid tumors. Some of these agents are currently in clinical trials.

Cytoplasmic signaling of CD47 can be mediated through its intracellular domain (ICD), although few proteins have so far been identified that direct interact with the CD47 cytoplasmic tail (Lamy L., J Biol Chem. 278:23915-21 (2003); Wu A. L., Mol Cell. 4:619-25 (1999)). Ubiquilin-1, one such binding partner, binds Gβγ and thereby tethers heterotrimeric G proteins to CD47 (N'Diaye E. N, J Cell Biol. 163:1157-65 (2003)). Ubiquilin-1 in this context inhibits chemotaxis signaled by the Gi-coupled receptor CXCR4 (Sick E., Glia. 59:308-19 (2011)). The foregoing are incorporated by reference herein in their entirety.

Human primary NK cells were shown to express SIRPα upon stimulation and bind to CD47. This reduces their killing efficacy for CD47-expressing cells. (See PCT/US20/39220, incorporated by reference herein in its entirety.)

Autologous induced pluripotent stem cells (iPSCs) theoretically constitute an unlimited cell source for patient-specific cell-based organ repair strategies. Their generation, however, poses technical and manufacturing challenges and is a lengthy process that conceptually prevents any acute treatment modalities. Allogeneic iPSC-based therapies or embryonic stem cell-based therapies are easier from a manufacturing standpoint and allow the generation of well-screened, standardized, high-quality cell products. Because pluripotent stem cells can be differentiated into any cell type of the three germ layers, the potential application of stem cell therapy is wide-ranging. Differentiation can be performed ex vivo or in vivo by transplanting progenitor cells that continue to differentiate and mature in the organ environment of the implantation site. Ex vivo differentiation allows researchers or clinicians to closely monitor the procedure and ensures that the proper population of cells is generated prior to transplantation. Because of their allogeneic origin, however, such cell products could undergo rejection.

### IV. SUMMARY OF THE INVENTION

The invention provides cells that have an increased immune checkpoint engagement function (Immune Checkpoint Engager “ICE” cells). In some embodiments, the ICE cells have a PD-1 engagement function (PD-1 engager cells). The ICE cells mitigate adaptive and innate immunity when transplanted into a subject when compared to a parental cell having a corresponding unmodified immune checkpoint engagement function. In some embodiments, the PD-1 engager cells are hypoimmune pluripotent (HIP) cells. In further embodiments, the HIP cells are blood type O (HIPO), Rhesus factor (Rh) negative (HIP−) or both type O and Rh− (HIPO−). In other embodiments, the PD-1 engager cells have been derived or differentiated from HIP, HIP−, or HIPO− cells.

Thus, the invention provides an Immune Checkpoint Engager (ICE) cell, comprising an engager molecule expressed on a cell surface, wherein the engager molecule engages with an immune checkpoint molecule on an immune cell, wherein the engager molecule is expressed at least at a level that protects the ICE cell from being killed by the immune cell. In some aspects, the engager molecule is a protein that does not engage SIRPα on the immune cell. In other aspects, the engager molecule engages PD-1.

In other aspects of the invention, the protein comprises an immunoglobulin domain that binds PD-1. In other aspects, the immunoglobulin domain is an antibody VH domain. In other aspects, the antibody VH domain comprises at least a 90% sequence identity to amino acid numbers 146-264 of SEQ ID NO:1. In preferred aspects, the antibody VH domain comprises the sequence of amino acid numbers 146-264 of SEQ ID NO:1. In other aspects, the immunoglobulin domain is an antibody VL domain. In other aspects, the antibody VL domain comprises at least a 90% sequence identity to amino acid numbers 21-130 of SEQ ID NO:1. In preferred aspects, the antibody VH domain comprises the sequence of amino acid numbers 21-130 of SEQ ID NO:1.

In some aspects of the invention, the ICE cell express a protein that is a fusion protein. In other aspects, the fusion protein comprises at least a 90% sequence identity to the IL-2 signal peptide of SEQ ID NO:2. In preferred aspects, the fusion protein comprises the IL-2 signal peptide of SEQ ID NO:2. In other aspects, the fusion protein comprises at least a 90% sequence identity to the CD8a hinge peptide of SEQ ID NO:3. In preferred aspects, the fusion protein comprises the CD8a hinge peptide of SEQ ID NO:3. In some aspects, the fusion protein comprises at least a 90% sequence identity to the PDGFR transmembrane domain (TMD) of SEQ ID NO:4. In preferred aspects, the fusion protein comprises the PDGFR TMD of SEQ ID NO:4.

In some aspects of the invention, the engager molecule is a protein having at least a 90% sequence identity to SEQ ID NO:1. In preferred aspects, the engager molecule has the sequence identity of SEQ ID NO:1.

In some aspects of the invention, the engager molecule comprises an antibody Fab or a single chain variable fragment (scFV) that binds to PD-1. In other aspects, the Fab or scFV binds to PD-1 with an affinity measured by its dissociation constant (Kd), wherein the Kd is between about 10⁻⁷ and 10⁻¹³ M. In other aspects, the engager molecule comprises one or more antibody complementarity determining regions (CDRs) that binds to PD-1. In other aspects, the one or more CDRs have at least a 90% sequence identity to any one of SEQ ID NOS: 18-23. In other aspects, the one or more CDRs have the sequence of any one of SEQ ID NOS: 18-23.

In some aspects of the invention, the engager molecule is a fusion protein comprising a heterologous transmembrane domain (TMD). In other aspects, the TMD comprises a single a helix, multiple a helices, or a rolled-up β sheet. In other aspects, the heterologous TMD is selected from the group consisting of CD85f, CD349, CD284, CD261, CD172b, CD277, CD186, CD156c, CD304, CD254, CD263, CD267, CD337, CD170, CD283, CD133, CD327, CD205, CD232, CD282, CD16b, CD85i, CD85a, CD85c, CD275, CD108, CD358, CD335, CD218b, CD355, CD336, CD160, CD25, CD4, CD8a, CD235a, CD233, CD230, CD90, CD74, CD3d, CD340, CD236, CD61, CD18, CD54, CD29, CD1a, CD5, CD220, CD2, CD66e, CD51, CD141, CD115, CD42b, CD221, CD271, CD55, CD243, CD98, CD10, CD41, CD14, CD45, CD228, CD16a, CD49e, CD126, CD63, CD48, CD7, CD140b, CD3g, CD117, CD28, CD8b, CD37, CD11b, CD107a, CD331, CD222, CD20, CD79a, CD32, CD143, CD324, CD42c, CD107b, CD56, CD102, CD49d, CD66a, CD142, CD59, CD62L, CD121a, CD122, CD13, CD155, CD119, CD19, CD116, CD46, CD1e, CD1d, CD227, CD44, CD62P, CD104, CD43, CD140a, CD31, CD152, CD326, CD62E, CD36, CD127, CD49b, CD105, CD35, CD223, CD138, CD325, CD58, CD106, CD53, CD120a, CD224, CD21, CD33, CD22, CD120b, CD11a, CD11c, CD363, CD73, CD88, CD204, CD332, CD9, CD203a, CD334, CD333, CD206, CD49f, CD238, CD252, CD89, CD124, CD181, CD182, CD24, CD95, CD40, CD49c, CD159a, CD159c, CD314, CD27, CD123, CD26, CD82, CD121b, CD34, CD38, CD30, CD1b, CD1c, CD154, CD6, CD52, CD132, CD32, CD66b, CD171, CD191, CD197, CD185, CD131, CD50, CD70, CD153, CD144, CD80, CD362, CD68, CD361, CD147, CD309, CD135, CD292, CD103, CD130, CD42d, CD66d, CD66c, CD96, CD110, CD79b, CD200, CD192, CD231, CD86, CD212, CD118, CD146, CD134, CD158a, CD158b1, CD158b2, CD158e, CD158k, CD158j, CD158i, CD178, CD295, CD151, CD97, CD183, CD39, CD239, CD193, CD194, CD195, CD196, CDw198, CDw199, CD296, CD298, CD49a, CD322, CD85g, CD184, CD172a, CD156a, CD339, CD156b, CD213a1, CD129, CD83, CD125, CD241, CD269, CD202b, CD87, CD164, CD136, CD137, CD249, CD69, CD91, CDw210b, CD167a, CD300c, CD157, CD317, CD148, CD161, CD215, CD150, CD11d, CD218a, CD210, CD166, CD162, CD213a2, CD242, CD158g, CD158h, CD279, CD111, CD281, CD226, CD234, CD167b, CD300e, CD276, CD305, CD300g, CD300d, CD109, CD272, CD163, CD302, CD158f1, CD85h, CD85d, CD177, CD158z, CD158f2, CD85j, CD300f, CD92, CD351, CD112, CD100, CD270, CD101, CD297, CD316, CD352, CD217, CD307b, CD307a, CD307c, CD307d, CD307e, CD114, CD180, CD158d, CD273, CD290, CD244, CD169, CD299, CD318, CD360, CD229, CD248, CD354, CD320, CD93, CD319, CD113, CD163b, CD289, CD288, CD329, CD274, CD353, CD172g, CD315, CD280, CD264, CD300a, CD312, CD84, CD344, CD350, CD246, CD201, CD338, CD208, CD257, CD328, CD286, CD357, CD294, CD321, CD265, CD278, ITGA7, ITGA8, ITGA9, ITGA10, ITGA11, CD51, CD41, CD29, CD18, CD61, and CD104. In other aspects, the TMD of the fusion protein is from CD47 (SEQ ID NO:5), CD64 (SEQ ID NO:6), or PDGFR (SEQ ID NO:4).

In other aspects, the TMD comprises a sequence with at least a 90% sequence identity to SEQ ID NO:5 or SEQ ID NO:6. In preferred aspects, the TMD comprises the sequence of SEQ ID NO:5 or SEQ ID NO:6. In other aspects, the TMD is from a 7 transmembrane protein (7TM) or an immunoglobulin cell-surface protein.

In some aspects of the invention, the engager molecule does not have an intracellular domain (ICD). In other aspects, the engager molecule has an intracellular domain from CD16, CD32, CD64, CD8, CD3, CD28, or CD137.

In some aspects of the invention, the engager molecule has one or more linker or hinge regions connecting ECD, TMD, or ICD sequences.

In other aspects, the protein is an antibody, receptor, ligand, or adhesion protein.

In some aspects of the invention, the ICE cell as described herein further comprises a reduced or eliminated HLA-I or HLA-II expression. In other aspects, the cell is ABO blood group type O. In other aspects, the cell is Rhesus factor negative (Rh−). In other aspects, the cell has a reduced or eliminated ABO blood group antigen selected from the group consisting of A1, A2, and B. In other aspects, the cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

In some aspects, the ICE cell of the invention is a hypoimmunogenic (HI) cell comprising: an endogenous Major Histocompatibility Complex Class I (HLA-I) function that is reduced when compared to an unmodified parental cell and an endogenous Major Histocompatibility Complex Class II (HLA-II) function that is reduced when compared to the unmodified parental cell.

In other aspects, the engager cell comprises modulated expression of one or more of HLA-I human leukocyte antigens, HLA-II human leukocyte antigens, CD64, CD47, CD38, CCR5, CXCR4, NLRC5, CIITA, B2M, HLA-A, HLA-B, HLA-C, HLA-E, HLA-G, PD-L1, CTLA-4-Ig, CD47, CI-inhibitor, IL-35, RFX-5, RFXAP, RFXANK, NFY-A, NFY-B, NFY-C, IRF-1, OX40, GITR, 4-1BB, CD28, B7-1, B7-2, ICOS, CD27, HVEM, SLAM, CD226, PD1, CTL4, LAG3, TIGIT, TIM3, CD160, BTLA, CD244, CD30, TLT, VISTA, B7-H3, PD-L2, LFA-1, CD2, CD58, ICAM-3, TCRA, TCRβ, FOXP3, HELIOS, ST2, PCSK9, APOC3, CD200, FASLG, CLC21, MFGE8, SERPIN B9, TGFβ, CD73, CD39, LAG3, IL1R2, ACKR2, TNFRSF22, TNFRSF23, TNFRS10, DAD1, PVR, or IFNγR1 d39 relative to a parental cell, wherein the engager cell is ABO blood group type O or Rhesus factor negative (Rh−).

In other aspects, the ICE cell of of the invention further comprises an elevated expression of an antibody Fc receptor on the cell surface, wherein the Fc receptor helps to evade antibody dependent cellular cytotoxicity (ADCC) or complement mediated cytotoxicity (CDC). In other aspects, the Fc receptor is CD16, CD32, CD64, or truncated CD64.

In some aspects, the ICE cell of the invention is pluripotent. In other aspects, the cell is a hypoimmune pluripotent (HIP) cell. In other aspects, the cell is a hypoimmune pluripotent cell having an ABO blood type O (HIPO). In other aspects, the cell is a hypoimmune pluripotent cell is Rh factor negative (HIP−). In other aspects, the cell is a hypoimmune pluripotent cell having an ABO blood type O and is Rh factor negative (HIPO−). In other aspects, the cell is a pluripotent (PSC) cell, induced PSC (iPSC), or an embryonic stem cell (ESC).

In some aspects of the invention, the ICE cell as described herein is a specific tissue type. In other aspects, the cell is a chimeric antigen receptor (CAR) cell, a T cell, an NK cell, an endothelial cell, a dopaminergic neuron, a cardiac cell, a pancreatic islet beta cell, a macrophage, a thyroid epithelial cell, a parathyroid cell, or a retinal pigment epithelium cell. In other aspects, the ICE cell of the invention is a CAR-T cell, a CAR-NK cell, a TCR T cell, or a TCR NK cell. In other aspects, the engager cell is differentiated from a pluripotent cell.

The invention provides a pharmaceutical composition comprising the ICE cell as described herein and a pharmaceutically-acceptable carrier.

The invention provides a medicament comprising the ICE cell as described herein and a pharmaceutically-acceptable carrier.

The invention provides a method of treating a disease in a subject, comprising transplanting the ICE cell as described herein into the subject. In other aspects, the disease is Type 1 diabetes, a cardiac disease, a neurological disease, an endocrine disease, cancer, blindness, or a vascular disease.

The invention provides a use of the ICE cells as described herein for preparing a pharmaceutical composition for treating a disease in a subject. In other aspects, the disease is Type 1 diabetes, a cardiac disease, a neurological disease, an endocrine disease, cancer, blindness, or a vascular disease.

In some aspects of the invention, the ICE cell as described herein, further comprises a Signal Regulatory Protein Alpha (SIRPα) engager molecule on the cell surface that engages with a SIRPα protein on the immune cell, wherein the SIRPα engagement prevents the engager cell from being killed by the immune cell, wherein the SIRPα engager cell surface molecule lacks a functional intracellular domain. In other aspects, the engager molecule is a protein. In other aspects, the protein is a fusion protein. In other aspects, the fusion protein comprises a CD47 extracellular domain (ECD). In other aspects, the engager molecule comprises an immunoglobulin superfamily domain. In other aspects, the SIRPα engager molecule comprises one or more antibody complementarity determining regions (CDRs) that binds to SIRPα. In other aspects, the CDRs comprise a sequence having at least a 90% sequence identity to any one of SEQ ID NOS:18-23. In other aspects, the CDRs comprise any one of the sequences of SEQ ID NOS: 18-23.

The invention provides an ICE cell as disclosed herein, wherein said engager molecule engages with one or more immune cell receptors selected from the group consisting of PD-1, TIM3, LILRB3, and LILRB1.

The invention provides an ICE cell as disclosed herein that further comprises a safety switch. In some aspects, the safety switch is a suicide gene. In other aspects, the suicide gene is a herpes simplex virus thymidine kinase gene (HSV-tk) that has a ganciclovir trigger. In other aspects, the suicide gene is an Escherichia coli cytosine deaminase gene (EC-CD) that has a 5-fluorocytosine (5-FC) trigger. In other embodiments, the safety switch is a protein tag expressed on the cell surface that is recognized by a clinical antibody to target the cell for destruction.

### V. BRIEF DESCRIPTION OF THE DRAWINGS

FIG. 1 shows an iPSC-derived endothelial cell (iEC) bioluminescence imaging (BLI) NK cell killing assay. Natural killer cells (NK cells) were stimulated with IL-2 for 72 h. The NK cells were then added to FLuc⁺ B2M^{-/-} CIITA^{-/-} (HLA class I and II double knockout, DKO) and B2M^{-/-} CIITA^{-/-} iECs additionally expressing the PD-1 engager (DKO PD-1 engager iECs). The drop of the BLI signal was assessed after 4 hours (mean±s.d., 3 independent experiments per group, bidirectional Student t-test). The PD-1 engager molecule conveyed protection for the iECs from NK cell killing.

FIG. 2 shows an iEC BLI CD8 T cell killing assay. Peripheral blood mononuclear cells (PBMCs) were primed with wt iECs in vitro and sorted CD3+CD8+ T cells were used as effector cells for this assay. CD3+CD8+ T cells were then added to FLuc⁺ wt and wt PD-1 engager iECs. The drop of BLI signal was assessed after 4 hours (mean±s.d., 3 independent experiments per group, bidirectional Student t-test). The PD-1 engager molecule protected the iECs from CD3+CD8+ T cell killing.

FIGS. 3A and 3B show in vitro impedance killing assays. The target cells are HLA-A2-positive human pancreatic beta cells. In the upper row of FIGS. 3A and 3B, the beta cells are unmodified. In the bottom row of FIGS. 3A and 3B, the beta cells express the PD-1 engager. Peripheral blood mononuclear cells (PBMCs) were primed with HLA-A2-positive wt iECs in vitro and sorted. CD3+CD8+ T cells were used as effector cells for this assay. When the primed CD3+CD8+ T cells were added to the assay at time point 0, the unmodified beta cells were killed, but the PD-1 engager-expressing human pancreatic beta cells survived (mean±s.d., 3 independent experiments per time point).

FIG. 4 shows the binding of recombinant PD-1 to DKO PD-1 engager iECs. Flow cytometry shows specific binding of PD-1 plus a secondary antibody. Secondary antibody only was used as a negative control.

FIG. 5: Human induced pluripotent stem cell (iPSC)-derived endothelial cells (iECs) were generated that express firefly luciferase and further contain B2M and CIITA double knockout (DKO). An exemplary TIM3 Engager protein linked to a red fluorescent protein (RFP) tag was transduced into these cells using Lentiviral particles. They were analyzed by flow cytometry. A histogram shows the RFP signal of untransduced iECs DKO (left peak) and transduced iECs DKO+TIM3-E (right peak), thus confirming their expression of the TIM3 Engager.

FIGS. 6A and 6B: Flow cytometry analysis for TIM3 surface expression on primary human peripheral blood NK cells and macrophages. 96.6% of NK cells were positive for TIM3 (FIG. 6A) and 67.2% of macrophages were positive for TIM3 (FIG. 6B).

FIGS. 7A and 7B: BLI killing assay over 2 hours with NK cells. Human induced pluripotent stem cell (iPSC)-derived endothelial cells (iECs) were generated that express firefly luciferase and further contain B2M and CIITA double knockout (DKO). These cells were plated with iECs DKO+TIM3-E and allowed to attach for 16 hours. Then, human peripheral blood NK cells were added to the wells and incubated for 2 hours. The percent BLI signal after the incubation is shown. The BLI signal for iECs DKO completely vanished (FIG. 7A). The BLI signal of iECs DKO+TIM3-E remained steady over the 2 hours (FIG. 7B).

FIGS. 8A and 8B: BLI killing assay over 2 hours with macrophages. Firefly luciferase-expressing human iECs DKO and iECs DKO+TIM3-E were plated and allowed to attach for 16 hours. Then, human peripheral blood macrophages were added to the wells and incubated for 2 hours. The percent BLI signal after the incubation is shown. The BLI signal for iECs DKO completely vanished (FIG. 8A). The BLI signal of iECs DKO+TIM3-E remained steady over the 2 hours (FIG. 8B).

FIGS. 9A and 9B: BLI killing assay over 24 hours with NK cells. Firefly luciferase-expressing human iECs DKO and iECs DKO+TIM3-E were plated and allowed to attach for 16 hours. Then, human peripheral blood NK cells were added to the wells and incubated for 24 hours. The percent BLI signal after the 24 hours NK incubation is shown. The BLI signal for iECs DKO completely vanished (FIG. 9A). The BLI signal of iECs DKO+TIM3-E remained steady over the 24 hours (FIG. 9B).

FIGS. 10A and 10B: BLI killing assay over 24 hours with macrophages. Firefly luciferase-expressing human iECs DKO and iECs DKO+TIM3-E were plated and allowed to attach for 16 hours. Then, human peripheral blood macrophages were added to the wells and incubated for 24 hours. The percent BLI signal after the 24 hours macrophage incubation is shown. The BLI signal for iECs DKO completely vanished (FIG. 10A). The BLI signal of iECs DKO+TIM3-E remained steady over the 24 hours and even increased somewhat (FIG. 10B).

FIG. 11: Human induced pluripotent stem cell (iPSC)-derived endothelial cells (iECs) were generated that express firefly luciferase and further contain B2M and CIITA double knockout (DKO). An exemplary LILRB3 Engager protein linked to a red fluorescent protein (RFP) tag was transduced into these cells using Lentiviral particles. They were analyzed by flow cytometry. A histogram shows the RFP signal of untransduced iECs DKO (left peak) and transduced iECs DKO+LILRB3-E (right peak), thus confirming their expression of the LILRB3 Engager.

FIGS. 12A and 12B: Flow cytometry analysis for LILRB3 surface expression on primary human peripheral blood NK cells and macrophages. 17.8% of NK cells were positive for LILRB3 (FIG. 12A) and 95.3% of macrophages were positive for LILRB3 (FIG. 12B).

FIGS. 13A and 13B: BLI killing assay over 2 hours with NK cells. Human induced pluripotent stem cell (iPSC)-derived endothelial cells (iECs) were generated that express firefly luciferase and further contain B2M and CIITA double knockout (DKO). These cells were plated and also iECs DKO+LILRB3-E that additionally express the LILRB3 Engager. Cells were allowed to attach for 16 hours. Then, human peripheral blood NK cells were added to the wells and incubated for 2 hours. The percent BLI signal after the incubation is shown. The BLI signal for iECs DKO completely vanished (FIG. 13A). The BLI signal of iECs DKO+LILRB3-E dropped markedly less over the 2 hours (FIG. 13B).

FIGS. 14A and 14B: BLI killing assay over 2 hours with macrophages. Firefly luciferase-expressing human iECs DKO and iECs DKO+LILRB3-E were plated and allowed to attach for 16 hours. Then, human peripheral blood macrophages were added to the wells and incubated for 2 hours. The percent BLI signal after the incubation is shown. The BLI signal for iECs DKO completely vanished (FIG. 14A). The BLI signal of iECs DKO+LILRB3-E dropped markedly less over the 2 hours (FIG. 14B).

FIGS. 15A and 15B: BLI killing assay over 24 hours with NK cells. Firefly luciferase-expressing human iECs DKO and iECs DKO+LILRB3-E were plated and allowed to attach for 16 hours. Then, human peripheral blood NK cells were added to the wells and incubated for 24 hours. The percent BLI signal after the 24 hours NK incubation is shown. The BLI signal for iECs DKO completely vanished (FIG. 15A). The BLI signal of iECs DKO+LILRB3-E dropped markedly less over the 24 hours (FIG. 15B).

FIGS. 16A and 16B: BLI killing assay over 24 hours with macrophages. Firefly luciferase-expressing human iECs DKO and iECs DKO+LILRB3-E were plated and allowed to attach for 16 hours. Then, human peripheral blood macrophages were added to the wells and incubated for 24 hours. The percent BLI signal after the 24 hours macrophage incubation is shown. The BLI signal for iECs DKO completely vanished (FIG. 16A). The BLI signal of iECs DKO+LILRB3-E dropped markedly less over the 24 hours and even increased somewhat (FIG. 16B).

FIG. 17: Human induced pluripotent stem cell (iPSC)-derived endothelial cells (iECs) were generated that express firefly luciferase and further contain B2M and CIITA double knockout (DKO). An exemplary LILRB1 Engager protein linked to a red fluorescent protein (RFP) tag was transduced into these cells using lentiviral particles. They were analyzed by flow cytometry. A histogram shows the RFP signal of untransduced iECs DKO (left peak) and transduced iECs DKO+LILRB1-E (right peak), thus confirming their expression of the LILRB1 Engager.

FIGS. 18A and 18B: Flow cytometry analysis for LILRB1 surface expression on primary human peripheral blood NK cells and macrophages. 75.8% of NK cells were positive for LILRB1 (FIG. 18A) and 99.9% of macrophages were positive for LILRB1 (FIG. 18B).

FIGS. 19A and 19B: BLI killing assay over 2 hours with NK cells. Human induced pluripotent stem cell (iPSC)-derived endothelial cells (iECs) were generated that express firefly luciferase and further contain B2M and CIITA double knockout (DKO). These cells were plated and also iECs DKO+LILRB1-E that additionally express the LILRB1 Engager. Cells were allowed to attach for 16 hours. Then, human peripheral blood NK cells were added to the wells and incubated for 2 hours. The percent BLI signal after the incubation is shown. The BLI signal for iECs DKO completely vanished (FIG. 19A). The BLI signal of iECs DKO+LILRB1-E remained steady over the 2 hours (FIG. 19B).

FIGS. 20A and 20B: BLI killing assay over 2 hours with macrophages. Firefly luciferase-expressing human iECs DKO and iECs DKO+LILRB1-E were plated and allowed to attach for 16 hours. Then, human peripheral blood macrophages were added to the wells and incubated for 2 hours. The percent BLI signal after the incubation is shown. The BLI signal for iECs DKO completely vanished (FIG. 20A). The BLI signal of iECs DKO+LILRB1-E remained steady over the 2 hours (FIG. 20B).

FIGS. 21A and 21B: BLI killing assay over 24 hours with NK cells. Firefly luciferase-expressing human iECs DKO and iECs DKO+LILRB1-E were plated and allowed to attach for 16 hours. Then, human peripheral blood NK cells were added to the wells and incubated for 24 hours. The percent BLI signal after the 24 hours NK incubation is shown. The BLI signal for iECs DKO completely vanished (FIG. 21A). The BLI signal of iECs DKO+LILRB1-E remained steady over the 24 hours (FIG. 21B).

FIGS. 22A and 22B: BLI killing assay over 24 hours with macrophages. Firefly luciferase-expressing human iECs DKO and iECs DKO+LILRB1-E were plated and allowed to attach for 16 hours. Then, human peripheral blood macrophages were added to the wells and incubated for 24 hours. The percent BLI signal after the 24 hours macrophage incubation is shown. The BLI signal for iECs DKO completely vanished (FIG. 22A). The BLI signal of iECs DKO+LILRB1-E remained steady over the 24 hours and even increased somewhat (FIG. 22B).

FIGS. 23A and 23B show that expression of the PD-1 Engager transgene is maintained during differentiation. iPSCs DKO were transduced to express the PD-1 Engager using lentiviral particles (iPSCs DKO+PD-1-E). Flow cytometry shows that robust expression can be achieved (FIG. 23A). When iPSCs DKO+PD-1-E were differentiated into iECs DKO+PD-1-E, transgene expression remained stable (FIG. 23B).

FIGS. 24A and 24B show that expression of the TIM3 Engager transgene is maintained during differentiation. iPSCs DKO were transduced to express the TIM3 Engager using lentiviral particles (iPSCs DKO+TIM3-E). Flow cytometry shows that robust expression can be achieved (FIG. 24A). When iPSCs DKO+TIM3-E were differentiated into iECs DKO+TIM3-E, transgene expression remained stable (FIG. 24B).

FIGS. 25A and 25B show that expression of the LILRB3 Engager transgene is maintained during differentiation. iPSCs DKO were transduced to express the LILRB3 Engager using lentiviral particles (iPSCs DKO+LILRB3-E). Flow cytometry shows that robust expression can be achieved (FIG. 25A). When iPSCs DKO+LILRB3-E were differentiated into iECs DKO+LILRB3-E, transgene expression remained stable (FIG. 25B).

FIGS. 26A and 26B show that expression of the LILRB1 Engager transgene is maintained during differentiation. iPSCs DKO were transduced to express the LILRB1 Engager using lentiviral particles (iPSCs DKO+LILRB1-E). Flow cytometry shows that robust expression can be achieved (FIG. 26A). When iPSCs DKO+LILRB1-E were differentiated into iECs DKO+LILRB1-E, transgene expression remained stable (FIG. 26B).

FIGS. 27A and 27B show the threshold for TIM3-E expression required to protect cells from NK cell killing. In order to assess whether there is a functional threshold for the expression of the TIM3-E to exert its protective effect from NK cell killing, iECs DKO were transduced with lentiviral particles carrying the TIM3-E transgene. After 3 days, the transduced pool of iECs was sorted into three populations based on their TIM3-E expression. The three populations are labeled iECs DKO+TIM3-E high, med, and low (FIG. 27A). For the subsequent BLI killing assay, the different populations were incubated with NK cells for 2 hours (FIG. 27B). All target cells were luciferase positive. The percent BLI signal after the 2 hours NK incubation is shown. The BLI signal for iECs DKO completely vanished. The BLI signals for iECs DKO+TIM3-E high and med remained stable, but the BLI signals for iECs DKO+TIM3-E low also dropped to the background. These results show that the functional threshold for the TIM3-E is between the low and med expression levels.

FIGS. 28A and 28B show the threshold for LILRB1-E expression required to protect cells from NK cell killing. In order to assess whether there is a functional threshold for the expression of the LILRB1-E to exert its protective effect from NK cell killing, iECs DKO were transduced with lentiviral particles carrying the LILRB1-E transgene. After 3 days, the transduced pool of iECs was sorted into three populations based on their LILRB1-E expression. The three populations are labeled iECs DKO+LILRB1-E high, med, and low (FIG. 28A). For the subsequent BLI killing assay, the different populations were incubated with NK cells for 2 hours (FIG. 28B). All target cells were luciferase positive. The percent BLI signal after the 2 hours NK incubation is shown. The BLI signal for iECs DKO completely vanished. The BLI signals for iECs DKO+LILRB1-E high and med remained stable, but the BLI signals for iECs DKO+LILRB1-E low also dropped to the background. These results show that the functional threshold for the LILRB1-E is between the low and med expression levels.

### VI. DETAILED DESCRIPTION OF THE INVENTION

The invention provides immune checkpoint engagement (ICE) cells that reduce immunity when transplanted into a subject when compared to a parental cell. In some embodiments the ICE cells have an increased Programmed Cell Death Protein 1 (PD-1) engagement function (PD-1 engager cells) that reduces immunity when transplanted into a subject when compared to a parental cell having an unmodified PD-1 engagement function. In some embodiments, the PD-1 engager cells are hypoimmune cells. In other embodiments, the cells are pluriopotent cells. In other embodiments, the cells are embryonic stem (ES) cells or induced pluripotent stem cells (iPSC). In other embodiments, the PD-1 engager cells are differentiated somatic cells. In other embodiments, the PD-1 engager cells are hypoimmune pluripotent (HIP) cells. In further embodiments, the HIP cells are blood type O (HIPO), Rhesus factor (Rh) negative (HIP−) or both type O and Rh− (HIPO−). In other embodiments, the PD-1 engager cells have been derived or differentiated from HIP, HIP−, or HIPO− cells. In other embodiments, the PD-1 engager cells comprise an antibody Fc receptor to protect against antibody dependent cellular cytotoxicity (ADCC) or complement dependent cytotoxicity (CDC). In other embodiments, the ICE cells express a ligand that binds to an inhibitory immune cell receptor selected from the group consisting of an LILRB1 engager, an LILRB3 engager, and a TIM3 engager. In other embodiments, the ICE cells are PD-1 engager cells that additionally express a ligand that binds to another inhibitory immune cell receptor selected from the group consisting of CD47, truncated CD47, an LILRB1 engager, an LILRB3 engager, a SIRPα engager, and a TIM3 engager.

It was surprising that agonists for PD-1, TIM3, LILRB3, and LILRB1 all inhibited immune effector cells to protect the therapeutic cells from cell killing. Thus, the instant invention recognized that immune checkpoint engagers, as a class, and not just individual members, are valid targets for agonists to inhibit immune effector cells. Furthermore, the invention now recognizes that this class of agonists must be expressed at threshold levels in order to protect ICE cells from immune cell killing.

The invention provides compositions and uses for PD-1 agonists to inhibit immune effector cells to generate cell therapeutics in regenerative medicine or immuneoncology with reduce host versus graft rejections. In some embodiments, sequence fragments from a PD-1 agonist antibody (U.S. Pat. No. 10,493,148, incorporated by reference herein in its entirety) were taken and implemented in a newly designed fusion protein (SEQ ID NO: 1). The fusion protein of this embodiment contains the following parts:
(IL-2 or CD8) signal peptide-VL-(GGGGS)3 linker-VH-CD8a hinge-PDGFR TMD

In other embodiments, the PD-1 engager cells have been derived or differentiated from the aforementioned cells. By way of example, the differentiated PD-1 engager cells may be endothelial cells, cardiomyocytes, hepatocytes, dopaminergic neurons, pancreatic islet cells, retinal pigment epithelium cells, and other cell types used for transplantation and medical therapies. These would include chimeric antigen receptor (CAR) cells and T cell receptor-engineered cells, such as CAR-T cells, TCR T cells, NK cells and CAR-NK cells.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “hypo-immunogenic” cell, “hypoimmune” cell, or “HI” cell herein is meant a cell that gives rise to a reduced immunological rejection response when transferred into an allogeneic host. In preferred embodiments, HI cells do not give rise to an immune response. Thus, “hypo-immunogenic” refers to a significantly reduced or eliminated immune response when compared to the immune response of a parental (i.e. “wt”) cell prior to immunoengineering.

By “hypo-immunogenic cell O−” “hypo-immunogenic ORh−” cell or “HIO−” cell herein is meant a HI cell that is also ABO blood group O and Rhesus Factor Rh−. HIO− cells may have been generated from O-cells, enzymatically modified to be O—, or genetically engineered to be O—.

By “HLA” or “human leukocyte antigen” complex herein is meant a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes HLA-A, HLA-B, HLA-C, and others. These present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes HLA-DP, HLA-DM, HLA-DOB, HLA-DQ and HLA-DR, and others that present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or non-human (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences. “β-2 microglobulin” or “β2M” or “B2M” protein refers to the human β2M protein that has the amino acid and nucleic acid sequences shown below; the human gene has accession number RefSeq NM 004048.4.

“CD47 protein” protein refers to the human CD47 protein that has the amino acid and nucleic acid sequences shown below; the human gene has accession number RefSeq NM_001777.4.

Upon ligation to the PD-1 receptor, the natural ligands PD-L1 or PD-L2 expressed on a cell may initiate downstream signaling in the cell with potentially unwanted perturbations of its physiology. To only achieve protection against immune cell killing, some aspects the invention separate the extracellular PD-1-binding function from intracellular signaling in the engineered cell using a PD-1 engager as described herein. In other aspects, the invention provides PD-1 engager fusion proteins with agonistic PD-1 binding activities but lacking unwanted intracellular signaling in the engineered cell. Other aspects provide a PD-1 engager fusion protein comprising a PD-L1 or PD-L2 extracellular domain (ECD). In some aspects of the invention, such a fusion protein would have a homologous or heterologous intracellular domain. In other aspects, the fusion protein would have a truncated intracellular domain or no intracellulard domain.

The invention provides a PD-1 engager fusion protein that is expressed on an engineered cell and designed to bind to PD-1 on an immune cell in an agonistic manner that activates PD-1 signaling. The effector immune cell can be any immune cell expressing PD-1 and can be from the myeloid lineage (e.g. monocytes, macrophages, or polymorphonuclear cells) as well as the lymphoid lineage (e.g. T cells, B cells, or NK cells).

The PD-1/PD-L1 pathway is an established immune checkpoint. Although the forced overexpression of PD-L1 on target cells can effectively induce an inhibitory signal in immune effector cells, PD-L1 can induce unwanted signaling in the engineered cells. The invention provides a novel strategy to utilize this immune checkpoint without perturbations of the physiology of the engineered cells. The PD-1 engager proteins disclosed herein do not have any intracellular signaling domain.

Thus, in some embodiments, the fusion proteins provided herein comprise an extracellular domain (ECD) and a transmembrane domain (TMD) and may or may not comprise an intracellular domain (ICD). The fusion protein typically does not have an ICD and is limited to an ECD and TMD.

In some aspects, the ECD comprises a PD-L1 or PD-L2 ECD, complementarity-determining regions (CDRs) of an agonistic anti-PD-1 antibody, or a single chain variable fragment (scFv) of an agonistic anti-PD-1 antibody. Regions of interest on the ECD include at least one CDR sequence, where a CDR may be 3, 4, 5, 6, 7, 8, 9, 10, 11, 12 or more amino acids. Alternatively, ECDs of interest contain more than one antibody variable regions. CDRs of anti-PD-1 antibodies are disclosed, for example, in U.S. Pat. No. 10,493,148, incorporated by reference herein in its entirety.

In some aspects, one or more residues of a sequence are altered to modify binding to achieve a more favored on-rate of binding, a more favored off-rate of binding, or both, such that an optimized binding is achieved.

In other aspects, the ECD contains linker regions or hinges connecting the sequences provided with either the TMD or with each other. In other aspects, modifications are made within one or more of the linker regions or hinge regions so long as these modifications do not eliminate the binding affinity of the fusion protein with PD-1.

In some aspects, an ECD has a contiguous sequence of at least about 10 amino acids as set forth in SEQ ID NO:1, at least about 15 amino acids, at least about 20 amino acids, at least about 25 amino acids, at least about 30 amino acids, up to the complete provided region. ECDs also include sequences that differ by up to 1, 2, 3, 4, 5, 6 or more amino acids as compared to the amino acids sequence set forth in SEQ ID NO:1. In other embodiments, an ECD has at least about an 80%, 85%, 90%, 95%, or about 99% sequence identity to the amino acid sequence set forth in SEQ ID NO:1.

Generally, the transmembrane domain (TMD) of the PD-1 engager fusion protein is not limited to a specific TMD sequence. Preferably, the TMD allows stable anchorage of the fusion protein in the membrane of a cell expressing the fusion protein (e.g. an endothelial cell, a cardiomyocyte, a pancreatic beta cell, a T cell, an NK cell, or a hematopoietic cell, etc). It further allows binding of the ECD to PD-1. In some aspects, the fusion protein does contain an ICD and binding to PD-1 allows signaling via the ICD. This might be beneficial for the engineered cell if such signaling enhances the intrinsic function of this cell. Enhanced functions can, for example, be achieved through enhanced adhesion via the activation of integrins. In other aspects, the fusion protein does not contain an ICD, but rather, is truncated after the TMD. In the latter case, binding of the fusion protein to PD-1 does not result in intracellular signaling in the engineered cell.

TMDs extend across the cell membrane lipid bilayer as a single a helix, as multiple a helices, or as a rolled-up β sheet. Some of these “single-pass” and “multipass” proteins have a covalently attached fatty acid chain inserted in the cytosolic lipid monolayer. Other membrane proteins are exposed at only one side of the membrane. Some of these are anchored to the cytosolic surface by an amphipathic a helix that partitions into the cytosolic monolayer of the lipid bilayer through the hydrophobic face of the helix. Others are attached to the bilayer solely by a covalently attached lipid chain-either a fatty acid chain or a prenyl group—in the cytosolic monolayer or, via an oligosaccharide linker, to phosphatidylinositol in the noncytosolic monolayer. (Alberts B, Johnson A, Lewis J, et al., Molecular Biology of the Cell. 4th edition. New York: Garland Science; ISBN-10: 0-8153-3218-1 (2002)).

In some aspects, an exemplary TMD of the immune checkpoint engager molecule is from CD85f, CD349, CD284, CD261, CD172b, CD277, CD186, CD156c, CD304, CD254, CD263, CD267, CD337, CD170, CD283, CD133, CD327, CD205, CD232, CD282, CD16b, CD85i, CD85a, CD85c, CD275, CD108, CD358, CD335, CD218b, CD355, CD336, CD160, CD25, CD4, CD8a, CD235a, CD233, CD230, CD90, CD74, CD3d, CD340, CD236, CD61, CD18, CD54, CD29, CD1a, CD5, CD220, CD2, CD66e, CD51, CD141, CD115, CD42b, CD221, CD271, CD55, CD243, CD98, CD10, CD41, CD14, CD45, CD228, CD16a, CD49e, CD126, CD63, CD48, CD7, CD140b, CD3g, CD117, CD28, CD8b, CD37, CD11b, CD107a, CD331, CD222, CD20, CD79a, CD32, CD143, CD324, CD42c, CD107b, CD56, CD102, CD49d, CD66a, CD142, CD59, CD62L, CD121a, CD122, CD13, CD155, CD119, CD19, CD116, CD46, CD1e, CD1d, CD227, CD44, CD62P, CD104, CD43, CD140a, CD31, CD152, CD326, CD62E, CD36, CD127, CD49b, CD105, CD35, CD223, CD138, CD325, CD58, CD106, CD53, CD120a, CD224, CD21, CD33, CD22, CD120b, CD11a, CD11c, CD363, CD73, CD88, CD204, CD332, CD9, CD203a, CD334, CD333, CD206, CD49f, CD238, CD252, CD89, CD124, CD181, CD182, CD24, CD95, CD40, CD49c, CD159a, CD159c, CD314, CD27, CD123, CD26, CD82, CD121b, CD34, CD38, CD30, CD1b, CD1c, CD154, CD6, CD52, CD132, CD32, CD66b, CD171, CD191, CD197, CD185, CD131, CD50, CD70, CD153, CD144, CD80, CD362, CD68, CD361, CD147, CD309, CD135, CD292, CD103, CD130, CD42d, CD66d, CD66c, CD96, CD110, CD79b, CD200, CD192, CD231, CD86, CD212, CD118, CD146, CD134, CD158a, CD158b1, CD158b2, CD158e, CD158k, CD158j, CD158i, CD178, CD295, CD151, CD97, CD183, CD39, CD239, CD193, CD194, CD195, CD196, CDw198, CDw199, CD296, CD298, CD49a, CD322, CD85g, CD184, CD172a, CD156a, CD339, CD156b, CD213a1, CD129, CD83, CD125, CD241, CD269, CD202b, CD87, CD164, CD136, CD137, CD249, CD69, CD91, CDw210b, CD167a, CD300c, CD157, CD317, CD148, CD161, CD215, CD150, CD11d, CD218a, CD210, CD166, CD162, CD213a2, CD242, CD158g, CD158h, CD279, CD111, CD281, CD226, CD234, CD167b, CD300e, CD276, CD305, CD300g, CD300d, CD109, CD272, CD163, CD302, CD158f1, CD85h, CD85d, CD177, CD158z, CD158f2, CD85j, CD300f, CD92, CD351, CD112, CD100, CD270, CD101, CD297, CD316, CD352, CD217, CD307b, CD307a, CD307c, CD307d, CD307e, CD114, CD180, CD158d, CD273, CD290, CD244, CD169, CD299, CD318, CD360, CD229, CD248, CD354, CD320, CD93, CD319, CD113, CD163b, CD289, CD288, CD329, CD274, CD353, CD172g, CD315, CD280, CD264, CD300a, CD312, CD84, CD344, CD350, CD246, CD201, CD338, CD208, CD257, CD328, CD286, CD357, CD294, CD321, CD265, CD278, ITGA7, ITGA8, ITGA9, ITGA10, ITGA11, CD51, CD41, CD29, CD18, CD61, and CD104. In other aspects, the TMD of the fusion protein is from CD47 (SEQ ID NO:5), CD64 (SEQ ID NO:6), or PDGFR (SEQ ID No:4).

In other aspects, the PD-1 engager fusion protein does not have an intracellular domain (ICD) to avoid signaling in the engineered cell. If considered beneficial, however, the ICD of the fusion protein can the ICDs from CD16, CD32, CD64, CD8, CD3, CD28, or CD137.

“CIITA protein” protein refers to the human CIITA protein that has the amino acid and nucleic acid sequences shown below; the human gene has the RefSeq accession number NM_000246.4.

By “wild type” in the context of a cell means a cell found in nature. However, in the context of cell therapeutics, as used herein, it also means that the cell may contain nucleic acid changes resulting in imortality or a de-differentiated state after reprograming but did not undergo the immune editing procedures of the invention to achieve hypo-immunogenicity, antibody evasion capacities, or express immune checkpoint engagers.

By “syngeneic” herein refers to the genetic similarity or identity of a host organism and a cellular transplant where there is immunological compatibility; e.g. no immune response is generated.

By “allogeneic” herein refers to the genetic dissimilarity of a host organism and a cellular transplant where an immune response is generated.

By “B2M−/−” herein is meant that a diploid cell has had the B2M gene inactivated in both chromosomes. As described herein, this can be done in a variety of ways.

By “PD-1 tg,” “PD-1 transgene,” or “PD-1+” herein is meant that the host cell expresses PD-1, in some cases by having at least one additional copy of the PD-1 gene or a PD-1 fusion protein gene.

By “CIITA−/−” herein is meant that a diploid cell has had the CIITA gene inactivated in both chromosomes. As described herein, this can be done in a variety of ways.

By “PD-L1 tg,” “PD-L1 transgene,” or “PD-L1+” herein is meant that the host cell expresses PD-L1, in some cases by having at least one additional copy of the PD-L1 gene or a PD-L1 fusion protein gene.

By “PD-L2 tg,” PD-L2 transgene.” or “PD-L2+” herein is meant that the host cell expresses PD-L2, in some cases by having at least one additional copy of the PD-L2 gene or a PD-L2 fusion protein gene.

By “CD47 tg,” “CD47 transgene,” or “CD47+” herein is meant that the host cell expresses CD47, in some cases by having at least one additional copy of the CD47 gene or a CD47 fusion protein gene.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” are agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” are agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least an 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at Ph 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (Ph 6.8), 0.1% sodium pyrophosphate, 5×Denhardt's solution, sonicated salmon sperm DNA (50 μl/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

As used herein, a “pharmaceutically acceptable carrier” or “therapeutic effective carrier” is aqueous or nonaqueous (solid), for example alcoholic or oleaginous, or a mixture thereof, and can contain a surfactant, emollient, lubricant, stabilizer, dye, perfume, preservative, acid or base for adjustment of pH, a solvent, emulsifier, gelling agent, moisturizer, stabilizer, wetting agent, time release agent, humectant, or other component commonly included in a particular form of pharmaceutical composition. Pharmaceutically acceptable carriers are well known in the art and include, for example, aqueous solutions such as water or physiologically buffered saline or other solvents or vehicles such as glycols, glycerol, and oils such as olive oil. A pharmaceutically acceptable carrier can contain physiologically acceptable compounds that act, for example, to stabilize or to increase the absorption of specific inhibitor, for example, carbohydrates, such as glucose, sucrose or dextrans, antioxidants such as ascorbic acid or glutathione, chelating agents, low molecular weight proteins or other stabilizers or excipients.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In some embodiments, an insertion, deletion, substitution, or other type of amino acid change in a PD-1 engager or a SIRPα engager protein. In some embodiments, the modification is in PD-1, PD-L1, PD-L2, SIRPα, CD47, CD16, CD32, CD64, truncated CD64, HSVtk, EC-CD, or iCasp9 variant polypeptide is prepared according to the methods described herein and known in the art. Such modifications differentiate them from the corresponding parent that has not been modified according to the methods described herein, such as wild-type proteins, naturally occurring mutant proteins, or other engineered proteins that do not include the modifications of such variant polypeptides. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell; e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene. In general, knock in technologies result in the integration of the extra copy of the transgene into the host cell.

### VII. CELLS OF THE INVENTION

The invention provides ICE cells. In some embodiments they are PD-1 engager cells. In other embodiments they are hypoimmune cells. Hypoimmune cells may include HIP cells, HIPO cells, HIPO− cells, and others. In other embodiments, the PD-1 engager cells are differentiated somatic cells. In other embodiments, the PD-1 engager cells are hypoimmune pluripotent (HIP) cells. In further embodiments, the HIP cells are blood type O (HIPO), Rhesus factor (Rh) negative (HIP−) or both type O and Rh− (HIPO−). In other embodiments, the PD-1 engager cells have been derived or differentiated from HIP, HIP−, or HIPO− cells. In other embodiments, the PD-1 engager cells comprise an antibody Fc receptor to protect against antibody dependent cellular cytotoxicity (ADCC) or complement dependent cytotoxicity (CDC).

The invention provides compositions and methodologies for generating a PD-1 engager cells. In some aspects, the cells are hypoimmune cells. In other aspects, the cells are differentiated somatic cells. In other aspects, the cells are pluripotent cells such as HIP cells, HIP− cells, HIPO− cells. In other aspects, the PD-1 engager cells are pluripotent (PSC) cells suitable for transplantion and/or differentiation. The PSC cells include induced PSCs (iPSC) or embryonic stem cells (ESC). In other aspects, the cells are of particular tissue types and have differentiated from the aforementioned PD-1 engager cells. By way of example, the differentiated PD-1 engager cells may be endothelial cells, cardiomyocytes, hepatocytes, dopaminergic neurons, pancreatic islet cells, retinal pigment epithelium cells, and other cell types used for transplantation and medical therapies. These would include chimeric antigen receptor (CAR) cells, such as CAR-T cells, CAR-NK cells, TCR T cells, TCR NK cells, TCR innate lymphoid cells (ILCs) and other engineered cell populations. See WO2018/132783, WO2020/018620, WO2020/018615, PCT/US2020/032272, and U.S. patent application Ser. Nos. 16/870,959, and 16/870,960, incorporated by reference herein in their entirety.

The invention provides PD-1 engager cells having PD-1 engager proteins that interact with PD-1 on the surface of T cells, NK cells, and other immune cells. The immune checkpoint engager molecules prevent cell killing and mitigate adaptive and innate immunity. In some embodiments, the PD-1 engager protein is an anti-PD-1 antibody tethered to the surface of the PD-1 engager cell. In some embodiments, the anti-PD-1 antibody is tethered via its fragment crystallizable (Fc) portion to a cell-surface. In other embodiments, the antigen-binding portion of the anti-PD-1 antibody (scFv) are bound to the cell surface via a transmembrane domain (TMD). In preferred embodiments, the TMD comprises one or more α-helices. In other preferred embodiments, the TMD is from a 7 transmembrane protein (7TM). In other preferred embodiments, the TMD is from an immunoglobulin cell-surface protein. In more preferred embodiments, the immunoglobulin cell-surface protein is an antibody, receptor, ligand, or adhesion protein. In some embodiments, the PD-1 engager cell results from a PD-L1 or PD-L2 fusion protein anchored onto the cell surface.

PD-1 engager protein expression may be accomplished in several ways as will be appreciated by those in the art using “knock in” or transgenic technologies. In some cases, PD-1 engager protein expression results from one or more transgenes.

Accordingly, in some embodiments, one or more copies of a PD-1 engager protein expression gene is added to the PD-1 engager cells under the control of an inducible or constitutive promoter, with the latter being preferred. In some embodiments, a lentiviral construct is employed as described herein or known in the art. The genes may integrate into the genome of the host cell under the control of a suitable promoter as is known in the art.

In some embodiments, the expression of the gene can be increased by altering the regulatory sequences of an endogenous gene locus, for example, by exchanging the endogenous promoter for a constitutive promoter or for a different inducible promoter. This can generally be done using known techniques such as CRISPR.

Once altered, the presence of sufficient PD-1 engager protein expression can be assayed using known techniques such as those described in the Examples, such as Western blots, ELISA assays or FACS assays using appropriate antibodies. In general, “sufficiency” in this context means an increase in PD-1 engager protein expression on the cell surface that silences NK cell killing.

Also within the scope of the invention are polypeptides that are antibodies. The term antibody is meant to include monoclonal antibodies, polyclonal antibodies, humanized antibodies, antibody fragments (e.g., Fc domains), Fab fragments, single chain antibodies, bi- or multi-specific antibodies, Llama antibodies, nano-bodies, diabodies, affibodies, Fv, Fab, F(ab′)2, Fab′, scFv, scFv-Fc, and the like. Also included in the term are antibody-fusion proteins, such as Ig chimeras. Preferred antibodies include humanized or fully human monoclonal antibodies or fragments thereof.

The terms “antibody” and “immunoglobulin” may include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The terms “full length antibody,” “intact antibody” and “whole antibody” are used herein interchangeably to refer to an antibody in its substantially intact form, not antibody fragments as defined below. The terms particularly refer to an antibody with heavy chains that contain the Fc region. “Antibody fragments” comprise a portion of an intact antibody, preferably comprising the antigen binding region thereof. Examples of antibody fragments include Fab, Fab′, F(ab′)2, and Fv fragments; diabodies; linear antibodies; single-chain antibody molecules; and multispecific antibodies formed from antibody fragments. The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies.

In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations that typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Antibodies that bind specifically to an antigen have a high affinity for that antigen. Antibody affinities may be measured by a dissociation constant (Kd). In certain embodiments, an antibody provided herein has a dissociation constant (Kd) of equal to or less than about 100 nM, 10 nM, 1 nM, 0.1 nM, 0.01 nM, or 0.001 nM (e.g. 10⁻⁷ M or less, from 10⁻⁷ M to 10⁻¹³ M, from 10⁻⁸ M to 10⁻¹³ M or from 10⁻⁹ M to 10⁻¹³ M).

In one embodiment, Kd is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20@) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using surface plasmon resonance assays using a BIACORE®-2000 or a BIACORER-3000 (BIAcore, Inc., Piscataway, N.J.) at 25° C. with, e.g., immobilized antigen CM5 chips at ˜10 response units (RU). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (70.2 μM) before injection at a flow rate of 5 μl/minute to achieve approximately 10 response units (RU) of coupled protein. Following the injection of antigen, 1 μM ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 I/min. Association rates (Kₒₙ) and dissociation rates (K_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. Other coupling chemistries for the target antigen to the chip surface (e.g., streptavidin/biotin, hydrophobic interaction, or disulfide chemistry) are also readily available instead of the amine coupling methodology (CM5 chip) described above, as will be understood by one of ordinary skill in the art.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al, Nature, 256: 495 (1975); Harlow et al, Antibodies: A Laboratory Manual, (Cold Spring Harbor Laboratory Press, 2nd ed. 1988); Hammerling et al., in: Monoclonal Antibodies and T-Cell Hybridomas pp. 563-681 (Elsevier, N.Y., 1981)), recombinant DNA methods (see, e.g., U.S. Pat. No. 4,816,567), phage display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004), and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; 5,661,016; Marks et al., Bio. Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996) and Lonberg and Huszar, Intern. Rev. Immunol. 13: 65-93 (1995). The above patents, publications, and references are incorporated by reference in their entirety.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit, or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications may be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23: 1035-1038 (1995); Hurle and Gross, Curr. Op. Biotech. 5:428-433 (1994). The foregoing references are incorporated by reference in their entirety.

A “human antibody” is one which comprises an amino acid sequence corresponding to that of an antibody produced by a human and/or has been made using any of the techniques for making human antibodies as disclosed herein. Such techniques include screening human-derived combinatorial libraries, such as phage display libraries (see, e.g., Marks et al., J. Mol. Biol, 222: 581-597 (1991) and Hoogenboom et al., Nucl. Acids Res., 19: 4133-4137 (1991)); using human myeloma and mouse-human heteromyeloma cell lines for the production of human monoclonal antibodies (see, e.g., Kozbor, J. Immunol, 133: 3001 (1984); Brodeur et al., Monoclonal Antibody Production Techniques and Applications, pp. 55-93 (Marcel Dekker, Inc., New York, 1987); and Boerner et al., J. Immunol, 147: 86 (1991)); and generating monoclonal antibodies in transgenic animals (e.g., mice) that are capable of producing a full repertoire of human antibodies in the absence of endogenous immunoglobulin production (see, e.g., Jakobovits et al., Proc. Natl. Acad. Sci USA, 90: 2551 (1993); Jakobovits et al., Nature, 362: 255 (1993); Bruggermann et al., Year in Immunol., 7: 33 (1993)). This definition of a human antibody specifically excludes a humanized antibody comprising antigen-binding residues from a non-human animal.

### A. Methodologies for Genetic Alterations

The invention includes methods of modifying nucleic acid sequences within cells or in cell-free conditions to generate PD-1 engager cells. Exemplary technologies include homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

As will be appreciated by those in the art, a number of different techniques can be used to engineer the modified cells of the invention, as well as the engineering them to become hypo-immunogenic as outlined herein.

In general, these techniques can be used individually or in combination. For example, in the generation of the PD-1 engager cells, CRISPR may be used to express PD-1 engager proteins such as anti-PD-1 immunoglobulins. In another example, viral techniques (e.g. lentivirus) are used to express PD-1 engager proteins.

a. CRISPR Technologies

In one embodiment, the cells are manipulated using clustered regularly interspaced short palindromic repeats)/Cas (“CRISPR”) technologies as is known in the art. CRISPR can be used to generate the PD-1 engager cells. There are a large number of techniques based on CRISPR, see for example Doudna and Charpentier, Science doi:10.1126/science.1258096, hereby incorporated by reference. CRISPR techniques and kits are sold commercially.

b. TALEN Technologies

In some embodiments, the cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

c. Zinc Finger Technologies

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

d. Viral Based Technologies

There are a wide variety of viral techniques that can be used to generate some embodiments of the PD-I engager cells of the invention including, but not limited to, the use of retroviral vectors, lentiviral vectors, adenovirus vectors and Sendai viral vectors. Episomal vectors used in the generation of ithe cells are described below.

For all of these technologies, well known recombinant techniques are used, to generate recombinant nucleic acids as outlined herein. In certain embodiments, the recombinant nucleic acids that encode a PD-1 engager protein, e.g. an anti-PD-1 immunoglobulin, may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable mammalian promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, the early promoter of human Cytomegalovirus (CMV), the eukaryotic translation elongation factor 1α (EF-1α), and the chicken β-Actin promoter coupled with CMV early enhancer (CAG). Examples of other heterologous mammalian promoters are the actin, immunoglobulin or heat shock promoter(s).

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

In some embodiments, the PD-1 engager cells are derived from stem cells.

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells (ESC) and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.)

“Pluripotent stem cells” as used herein have the potential to differentiate into any of the three germ layers: endoderm (e.g. the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells, and “miPSCs” are murine induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. As described herein, cells do not need to pass through pluripotency to be reprogrammed into endodermal progenitor cells and/or hepatocytes.

### B. Generation of Hypo-Immunogenic PD-1 Engager Cells

Generating HI cells is done with as few as three genetic changes, resulting in minimal disruption of cellular activity but conferring immunosilencing to the cells. The techniques are disclosed in WO2018/132783, WO2020/018620, WO2020/018615, PCT/US2020/032272, and U.S. patent application Ser. Nos. 16/870,959, and 16/870,960, incorporated by reference herein in their entirety. The techniques are discussed briefly below.

As discussed herein, one embodiment utilizes a reduction or elimination in the protein activity of MHC I and II (HLA I and II when the cells are human). This can be done by altering genes encoding their components. In one embodiment, the coding region or regulatory sequences of the gene are disrupted using CRISPR. In another embodiment, gene translation is reduced using interfering RNA technologies. Another embodiment is a change in a gene that regulates susceptibility to macrophage phagocytosis. This may be a “knock in” of a gene using viral technologies.

### 1. HLA-I Reduction

The HI PD-1 engager cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the PD-1 engager cells can be measured using techniques known in the art and as described below; for example, FACS techniques using labeled antibodies that bind the HLA complex; for example, using commercially available HLA-A,B,C antibodies that bind to the the alpha chain of the human major histocompatibility HLA Class I antigens.

a. B2M Alteration

In one embodiment, the reduction in HLA-I activity is done by disrupting the expression of the β-2 microglobulin gene in the HI PD-1 engager cell, as disclosed herein. This alteration is generally referred to herein as a gene “knock out”, and in the cells of the invention it is done on both alleles in the host cell. Generally, the techniques to do both disruptions is the same.

A particularly useful embodiment uses CRISPR technology to disrupt the gene. Another embodiment uses programmable transcriptional memory by CRISPR-based epigenome editing (Nuñez J K, Cell. 184:2503-2519 (2021), incorporated by reference herein in its entirety). In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2M gene in mouse or the B2M gene in human. After gene editing, the transfected PD-1 engager cell cultures are dissociated to single cells. Single cells are expanded to full-size colonies and tested for CRISPR edit by screening for presence of aberrant sequence from the CRISPR cleavage site. Clones with deletions in both alleles are picked. Such clones did not express B2M as demonstrated by PCR and did not express HLA-I as demonstrated by FACS analysis.

Assays to test whether the B2M gene has been inactivated are known and described herein. In one embodiment, the assay is a Western blot of cells lysates probed with antibodies to the B2M protein. In another embodiment, reverse transcriptase polymerase chain reactions (rt-PCR) confirms the presence of the inactivating alteration.

In addition, the cells can be tested to confirm that the HLA I complex is not expressed on the cell surface. This may be assayed by FACS analysis using antibodies to one or more HLA cell surface components as discussed above.

### 2. HLA-II Reduction

In some embodiments, in addition to a reduction in HLA I, the HI PD-1 engager cells of the invention may also lack MHC II function (HLA II from human-derived cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II (HLA II) function in the PD-1 engager cells or their derivatives can be measured using techniques known in the art such as Western blotting using antibodies to the protein, FACS techniques, rt-PCR techniques, etc.

a. CIITA Alteration

In one embodiment, the reduction in HLA-II activity is done by disrupting the expression of the CIITA gene in the PD-1 engager cell, as shown herein. This alteration is generally referred to herein as a gene “knock out”, and in the PD-1 engager cells of the invention it is done on both alleles in the host cell.

Assays to test whether the CIITA gene has been inactivated are known and described herein. In one embodiment, the assay is a Western blot of cells lysates probed with antibodies to the CIITA protein. In another embodiment, reverse transcriptase polymerase chain reactions (rt-PCR) confirms the presence of the inactivating alteration.

In addition, the cells can be tested to confirm that the HLA II complex is not expressed on the cell surface. Again, this assay is done as is known in the art. Exemplary analyses include Western Blots or FACS analysis using commercial antibodies that bind to human HLA Class II HLA-DR, DP and most DQ antigens as outlined below.

A particularly useful embodiment uses CRISPR technology to disrupt the CIITA gene. CRISPRs ae designed to target the coding sequence of the CIITA gene, an essential transcription factor for all MHC II molecules. After gene editing, the transfected cell cultures are dissociated into single cells. They are expanded to full-size colonies and tested for successful CRISPR editing by screening for the presence of an aberrant sequence from the CRISPR cleavage site. Clones with deletions that do not express CIITA are determined by PCR and may be shown not to express MHC II/HLA-II by FACS analysis. Another embodiment uses programmable transcriptional memory by CRISPR-based epigenome editing.

### 3. Blood Type O Rh Negative Cells

Blood products can be classified into different groups according to the presence or absence of antigens on the surface of every red blood cell in a person's body (ABO Blood Type). The A, B, AB, and A1 antigens are determined by the sequence of oligosaccharides on the glycoproteins of erythrocytes. The genes in the blood group antigen group provide instructions for making antigen proteins. Blood group antigen proteins serve a variety of functions within the cell membrane of red blood cells. These protein functions include transporting other proteins and molecules into and out of the cell, maintaining cell structure, attaching to other cells and molecules, and participating in chemical reactions.

The Rhesus Factor (Rh) blood group is the second most important blood group system, after the ABO blood group system. The Rh blood group system consists of 49 defined blood group antigens, among which five antigens, D, C, c, E, and e, are the most important. Rh(D) status of an individual is normally described with a positive or negative suffix after the ABO type. The terms “Rh factor,” “Rh positive,” and “Rh negative” refer to the Rh(D) antigen only. Antibodies to Rh antigens can be involved in hemolytic transfusion reactions and antibodies to the Rh(D) and Rh(c) antigens confer significant risk of hemolytic disease of the fetus and newborn. ABO antibodies develop in early life in every human. However, rhesus antibodies in Rh− humans develop only when the person is sensitized. This occurs by giving birth to a rh+ baby or by receiving an Rh+ blood transfusion.

This invention provides PD-1 engager cells having an ABO blood type O and/or Rhesus Factor negative (O−) populations of pluripotent (PSCO−) cells suitable for transplantion and/or differentiation. The PSCO− cells include induced iPSCs (iPSCO−), embryonic ESCs (ESCO−), and cells differentiated from those cells, including O− endothelial cells, O− cardiomyocytes, O− hepatocytes, O− dopaminergic neurons, O− pancreatic islet cells, O− retinal pigment epithelium cells, and other O− cell types used for transplantation and medical therapies. These would include O− chimeric antigen receptor (CAR) cells, such as CAR-T cells, CAR-NK cells, and other engineered cell populations. In some embodiments, the cells are not hematopoietics stem cells. The invention further provides universally acceptable “off-the-shelf” ESCO−s and PSCO−s and derivatives thereof for generating or regenerating specific tissues and organs.

Another aspect of the invention provides methods of generating populations of PSCO−, iPSCO−, ESCO− and other O− cells for transplantation. The invention also provides methods of treating diseases, disorders, and conditions that benefit from the transplantation of pluripotent or differentiated cells.

In some embodiments of the invention, the ABO blood group type O results from a reduced ABO blood group protein expression. In other aspects, the ABO blood group is endogenously type O. In some aspects of the invention, the HIPO− cell has an ABO blood group type O that results from a disruption in human Exon 7 of the ABO gene. In some embodiments, both alleles of Exon 7 of the ABO gene are disrupted. In some embodiments, the disruption in both alleles of Exon 7 of the ABO gene results from a Clustered Regularly Interspaced Short Palindromic Repeats (CRISPR)/Cas9 reaction that disrupts both of the alleles. Another embodiment uses programmable transcriptional memory by CRISPR-based epigenome editing to inactivate this gene.

In other aspects, the ABO blood group type O results from an enzymatic modification of an ABO gene product on a surface of the cell. In a preferred aspect, the enzymatic modification removes a carbohydrate from the ABO gene product. In another preferred aspect, the enzymatic modification removes a carbohydrate from an ABO A1 antigen, A2 antigen, or B antigen.

In some embodiments of the invention, the Rh blood group is endogenously type Rh−. In another aspect, the Rh− blood group results from reducing or eliminating Rh protein expression. In another aspect, the type Rh− results from disrupting the gene encoding Rh C antigen, Rh E antigen, Kell K antigen (KEL), Duffy (FY) Fya antigen, Duffy Fy3 antigen, Kidd (JK) Jkb antigen, or/and or Kidd SLC14A1. In some embodiments the disruption results from a CRISPR/Cas9 reaction that disrupts both alleles of the gene encoding Rh C antigen, Rh E antigen, Kell K antigen (KEL), Duffy (FY) Fya antigen, Duffy Fy3 antigen, Kidd (JK) Jkb antigen, or/and or Kidd SLC14A1.

In some embodiments of the invention, the O− cells (e.g., PSCO−, iPSCO−, ESCO− and cells derived therefrom) of the invention are of mammalian origin, for example, human, bovine, porcine, chicken, turkey, horse, sheep, goat, donkey, mule, duck, goose, buffalo, camel, yak, llama, alpaca, mouse, rat, dog, cat, hamster, or guinea pig origin.

In a specific embodiment, the invention provides hypoimmune PD-1 engager cells with an ABO blood type O Rhesus Factor negative (HIPO−) cells that evade rejection by the host allogeneic immune system and avoid blood antigen type rejection. In some embodiments, the HIPO− cells are engineered to reduce or eliminate HLA-I and HLA-II expression, increase expression of an endogenous protein that reduces the susceptibility of the pluripotent cell to macrophage phagocytosis, and comprise a universal blood group O Rh− (“O−”) blood type. The universal blood type may be achieved by eliminating ABO blood group A and B antigens and Rh factor expression, or by starting with an O− cell line. These novel HIPO− cells evade host immune rejection because they have an impaired antigen presentation capacity, protection from innate immune clearance, and lack blood group rejection.

### 4. Suicide Genes

In some embodiments, the invention provides HI PD-1 engager cells that comprise a “suicide gene” or “suicide switch”. These are incorporated to function as a “safety switch” that can cause the death of the cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10):1932-1943 (2012), Xu et al., Cell Res. 8:73-8 (1998), both incorporated herein by reference in their entirety.

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al, N. Engl. J. Med 365; 18 (2011); Tey et al., Biol. Blood Marrow Transplant. 13:913-924 (2007), each of which are incorporated by reference herein in their entirety.)

In other embodiments, a binding epitope for existing CAR T cells is included into the extracellular structure of the Engager cell as safety strategy. Such a binding epitope can be CD19, the CD19 ectodomain, or specific anti-CD19 binding regions from, e.g, CAR T cells FMC63, 4G7-2E3 or 3B10. (See Klesmith J R, et al., Biochemistry. 3; 58(48):4869-4881 (2019), incorporated by reference herein in its entirety.) To eliminate the PD-1-Engager cells, a CAR T cell targeting the incorporated anti-CD19 binding region is used. The CAR T cells detect the incorporated binding epitope and kill the Engager cell. Binding regions can be chosen from other CAR T cell targets such as CD20, CD22, CD38, CD123, CS1, CD171, BCMA, MUC16, ROR1, AXL 2, B7-H3, CD147, CD171, CD20, CD44v6, CD70, CEA, CLDN18.2, CLDN6, DLL3, DR5, EGFR, EGFRvIII, EpCAM, ErbB, FRα, GD2, gp100, GPC3, HER2, IL-13Rα2, LFA1, MMP2, MSLN, MUC1, MUC1*, MUC16ecto, NECTIN4, NKG2D, NKG2DL, PSCA, PSMA, ROR2, TM4SF1, TnMUCI, or WT1.

### 5. Fc Sequestration

If an antibody binds to an unprotected cell via its Fab regions, the Fc can be bound by NK cells (mostly via their CD16 receptor), macrophages (mostly via CD16, CD32, or CD64), B-cells (mostly via CD32), or granulocytes (mostly via CD16, CD32, or CD64). These can mediate antibody-dependent cellular cytotoxicity (ADCC). If complement binds to the Fe, it can cause complement dependent cytotoxicity (CDC).

In some embodiments, the PD-1 engager cells of the invention comprise elevated levels of receptors that recognize the Fc portion of IgG. Receptors that recognize the Fc portion of IgG are divided into four different classes: FcγRI (CD64), FcγRII (CD32), FcγRIII (CD16), and FcγRIV. This reduces the propensity for the cell transplant recipient's immune system to reject allogeneic material. The cells expressing elevated CD16, CD32, CD64, or truncated CD64 evade ADCC or CDC. Fe Sequestration is disclosed in WO2021076427, incorporated by reference herein in its entirety.

### 6. SIRPα Engager Cells

In some embodiments, the cells of the invention additionally have an increased Signal Regulatory Protein Alpha (SIRPα) engagement function (PD-1/SIRPα engager cells) that resist innate immunity when transplanted into a subject when compared to a parental cell having an unmodified SIRPα engagement function. In some embodiments, the PD-1/SIRPα engager cells are hypoimmune cells. In other embodiments, the PD-1/SIRPα engager cells are differentiated somatic cells. In other embodiments, the PD-1/SIRPα engager cells are pluripotent cells or hypoimmune pluripotent (HIP) cells. In further embodiments, the HIP cells are blood type O (HIPO), Rhesus factor (Rh) negative (HIP−) or both type O and Rh− (HIPO−). In other embodiments, the PD-1/SIRPα engager cells have been derived or differentiated from HIP, HIP−, or HIPO− cells. In other embodiments, the PD-1/SIRPα engager cells comprise an antibody Fc receptor to protect against antibody dependent cellular cytotoxicity (ADCC) or complement dependent cytotoxicity (CDC). SIRPα engager cells are described in PCT/US21/62008, incorporated by reference herein in its entirety.

### 7. Assays for HI Phenotypes

Once the HI cells have been generated, they may be assayed for their hypo-immunogenicity as is generally described herein.

For example, hypo-immunogenicity are assayed using a number of techniques. One exemplary technique includes transplantation into allogeneic hosts and monitoring for HI PD-1 engager cell survival. The cells may be transduced to express luciferase and can then be followed using bioluminescence imaging. Similarly, the T cell or B cell response of the host animal to the HI PD-1 engager cells are tested to confirm that they do not cause an immune reaction in the host animal. T cell function is assessed by Elispot, Elisa, FACS, PCR, or mass cytometry (CYTOF). B cell response or antibody response is assessed using FACS or luminex. Additionally, or alternatively, the cells may be assayed for their ability to avoid innate immune responses, e.g. NK cell killing. NK cell cytolytic activity is assessed in vitro or in vivo using techniques known in the art.

### C. Generation of Hypoimmune (HI) O− PD-1 Engager Cells

In some aspects of the invention, the PD-1 engager cells generated as above will already be ABO blood group O and Rh factor negative (−) cells because the process will have started with NK cells having an O− blood type.

Other aspects of the invention involve the enzymatic conversion of A and B antigens. In preferred aspects, the B antigen is converted to O using an enzyme. In more preferred aspects, the enzyme is an α-galactosidase. This enzyme eliminates the terminal galactose residue of the B antigen. Other aspects of the invention involve the enzymatic conversion of A antigen to O. In preferred aspects, the A antigen is converted to O using an α-N-acetylgalactosaminidase. Enzymatic conversion is discussed, e.g., in Olsson et al., Transfusion Clinique etBiologique 11:33-39 (2004); U.S. Pat. Nos. 4,427,777, 5,606,042, 5,633,130, 5,731,426, 6,184,017, 4,609,627, and 5,606,042; and Int'l Pub. No. WO9923210, each of which are incorporated by reference herein in their entirety.

Other embodiments of the invention involve genetically engineering the cells by knocking out the ABO gene Exon 7 or silencing the SLC14A1 (JK) gene. Other embodiments of the invention involve knocking out the C and E antigens of the Rh blood group system (RH), K in the Kell system (KEL), Fya and Fy3 in the Duffy system (FY), Jkb in the Kidd system (JK), or U and S in the MNS blood group system. Any knockout methodology known in the art or described herein, such as CRISPR, talens, or homologous recombination, may be employed.

Techniques for generating hypoimmune ABO blood group O Rh Factor (−) cells are described in Provisional App. No. 62/846,399 which is incorporated by reference herein in its entirety.

### D. Embodiments of the Invention

The PD-I engager cells, or derivatives thereof, of the invention may be used to treat, for example, Type 1 diabetes, cardiac diseases, neurological diseases, cancer, blindness, vascular diseases, and other diseases/disorders that respond to regenerative medicine therapies. In particular, the invention contemplates using the PD-I engager cells for differentiation into any cell type. Thus, provided herein are iPSC, ESC, HIP, iPSCO, ESCO, HIPO, iPSCO−, ESCO−, and HIPO− PD-1 engager cells, or derivatives or differentiated cells thereof, that exhibit pluripotency but do not result in a host immune response or a markedly mitigated immune response when transplanted into an allogeneic host such as a human patient.

In one aspect, the present invention provides a PD-I engager cell, or derivative thereof, comprising a nucleic acid encoding a chimeric antigen receptor (CAR), wherein endogenous β-2 microglobulin (B2M) gene activity and endogenous class II transactivator (CIITA) gene activity have been eliminated and a PD-1 engager molecule is provided on the cell surface. The CAR can comprise an extracellular domain, a transmembrane domain, and an intracellular signaling domain. In some embodiments, the extracellular domain binds to an antigen selected from the group consisting of CD19, CD20, CD22, CD38, CD123, CS1, CD171, BCMA, MUC16, ROR1, WT1, AXL 2. B7-H3, CD147, CD171, CD20, CD44v6, CD70, CEA, CLDN18.2, CLDN6, DLL3, DR5, EGFR, EGFRvIII, EpCAM, ErbB, FRα, GD2, gp100, GPC3, HER2, IL-13Rα2, LFA1, MMP2, MSLN, MUC1, MUC1*, MUC16ecto, NECTIN4, NKG2D, NKG2DL, PSCA, PSMA, ROR2, TM4SF1, and TnMUCI. In certain embodiments, the extracellular domain comprises a single chain variable fragment (scFv). In some embodiments, the transmembrane domain comprises CD3ζ, CD4, CD8α, CD28, 4-1BB, OX40, ICOS, CTLA-4, PD-1, LAG-3, CD64, PDGF, and BTLA. In certain embodiments, the intracellular signaling domain comprises CD3ζ, CD28, 4-1BB, OX40, ICOS, CTLA-4, PD-1, LAG-3, and BTLA.

In certain embodiments, the CAR comprises an anti-CD19 scFv domain, a CD28 transmembrane domain, and a CD3 zeta signaling intracellular domain. In some embodiments, the CAR comprises an anti-CD19 scFv domain, a CD28 transmembrane domain, a 4-1BB signaling intracellular domain, and a CD3 zeta signaling intracellular domain.

In another aspect of the invention, provided is an isolated PD-1 engager CAR-T cell or hypoimmune CAR-T cell produced by in vitro differentiation of any one of the pluripotent cells described herein. In some embodiments, the CAR-T cell is a cytotoxic HIPO− CAR-T cell.

In some aspects, the invention provides a PD-1 engager NK or CAR-NK cell, or an iPSC-derived NK cell.

In various embodiments, the in vitro differentiation comprises culturing the PD-1 engager cell, or derivative thereof, carrying a CAR construct in a culture media comprising one or more growth factors or cytokines selected from the group consisting of bFGF, EPO, Flt3L, IGF, IL-3, IL-6, IL-15, GM-CSF, SCF, and VEGF. In some embodiments, the culture media further comprises one or more growth factors or cytokines selected from the group consisting of a BMP activator, a GSK3 inhibitor, a ROCK inhibitor, a TGFβ receptor/ALK inhibitor, and a NOTCH activator.

In particular embodiments, the isolated PD-1 engager CAR-T or CAR-NK cells are produced by in vitro differentiation of any one of iPSC, ESC, HIP, iPSCO, ESCO, HIPO, iPSCO−, ESCO−, or HIPO− PD-1 engager cells carrying the CAR-T constructs. In other embodiments, they are used to treat cancer.

In another aspect of the invention, provided is a method of treating a patient with cancer by administering a composition comprising a therapeutically effective amount of any of the isolated PD-1 engager CAR-T CAR-NK cells described herein. In some embodiments, the composition further comprises a therapeutically effective carrier.

In some embodiments, the administration step comprises intravenous administration, subcutaneous administration, intranodal administration, intratumoral administration, intrathecal administration, intrapleural administration, and intraperitoneal administration. In certain instances, the administration further comprises a bolus or by continuous perfusion.

In some embodiments, the cancer is a blood cancer selected from the group consisting of leukemia, lymphoma, and myeloma. In various embodiments, the cancer is a solid tumor cancer or a liquid tumor cancer.

In another aspect, the present invention provides a method of making any one of the isolated PD-1 engager CAR-T CAR-NK cells described herein. The method includes in vitro differentiating of any one of the iPSC, ESC, HIP, iPSCO, ESCO, HIPO, iPSCO−, ESCO−, or HIPO− PD-1 engager cells of the invention. In vitro differentiation may comprise culturing the cells in a culture media comprising one or more growth factors or cytokines selected from the group consisting of bFGF, EPO, Flt3L, IGF, IL-2, IL-3, IL-6, IL-7, IL-15, GM-CSF, SCF, and VEGF. In some embodiments, the culture media further comprises one or more growth factors or cytokines selected from the group consisting of a BMP activator, a GSK3 inhibitor, a ROCK inhibitor, a TGFβ receptor/ALK inhibitor, and a NOTCH activator.

In some embodiments, the in vitro differentiating comprises culturing the iPSC, ESC, HIP, iPSCO, ESCO, HIPO, iPSCO−, ESCO−, or HIPO− PD-1 engager cells on feeder cells. In various embodiments, the in vitro differentiating comprises culturing in simulated microgravity. In certain instances, the culturing in simulated microgravity is for at least 72 hours.

In some aspects, provided herein is an isolated, engineered hypoimmune cardiac cell (hypoinmmuinogenic cardiac cell), for example a cardiomyocyte, differentiated from an iPSC, ESC, HIP, iPSCO, ESCO, HIPO, iPSCO−, ESCO−, or HIPO− PD-1 engager cell.

Cardiomyocytes were previously thought to lack ABO blood group antigens. Differentiation of an ABO blood group type B human embryonic stem cell line into cardiomyocyte-like cells was observed to result in the loss of the B antigen, suggesting that loss of these antigens may occur early during human embryogenesis. See, e.g., Mölne et al., Transplantation. 86(10):1407-13 (2008), incorporated by reference herein in its entirety. Other studies also reported that differentiation of induced human pluripotent stem cells into cardiomyocyte-like cells caused the progressive loss of the ABO blood group type A antigen in these cells. See, e.g., Saljö et al., Scientific Reports. 13072: 1-14 (2017). Surprisingly, however, the inventors determined that cardiomyocytes express ABO blood group antigens that can cause rejection of such cells to an unmatched recipient.

Accordingly, in some aspects, provided herein is a method of treating a patient suffering from a heart condition or disease. The method comprises administering a composition comprising a therapeutically effective amount of a population of any one of the isolated PD-1 engager cardiac cells derived from iPSC, ESC, HIP, iPSCO, ESCO, HIPO, iPSCO−, ESCO−, or HIPO− PD-1 engager cells as described herein. In some embodiments, the composition further comprises a therapeutically effective carrier.

In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the heart condition or disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, other cardiomyopathy, myocarditis, myocardial ischemic reperfusion injury, ventricular dysfunction, heart failure, congestive heart failure, coronary artery disease, end stage heart disease, atherosclerosis, ischemia, hypertension, restenosis, angina pectoris, rheumatic heart, arterial inflammation, or cardiovascular disease.

In some aspects, provided herein is a method of producing a population of cardiac cells from a population of PD-1 engager cells by in vitro differentiation, wherein endogenous β-2 microglobulin (B2M) gene activity and endogenous class II transactivator (CIITA) gene activity have been eliminated and a PD-1 engager molecule is provided on the cell surface. The method comprises: (a) culturing a population of PD-1 engager cells in a culture medium comprising a GSK inhibitor; (b) culturing the population of PD-1 engager cells in a culture medium comprising a WNT antagonist to produce a population of pre-cardiac cells; and (c) culturing the population of pre-cardiac cells in a culture medium comprising insulin to produce a population of O− hypoimmune cardiac cells. In some embodiments, the GSK inhibitor is CHIR-99021, a derivative thereof, or a variant thereof. In some instances, the GSK inhibitor is at a concentration ranging from about 2 μM to about 10 μM. In some embodiments, the WNT antagonist is IWR1, a derivative thereof, or a variant thereof. In some instances, the WNT antagonist is at a concentration ranging from about 2 μM to about 10 μM.

In some aspects, provided herein is an isolated, engineered PD-1 engager endothelial cell differentiated from an iPSC, ESC, HIP, iPSCO, ESCO, HIPO, iPSCO−, ESCO−, or HIPO− PD-1 engager cell. In other aspects, the isolated, engineered O− or O− hypoimmune endothelial cell is selected from the group consisting of a capillary endothelial cell, vascular endothelial cell, aortic endothelial cell, brain endothelial cell, and renal endothelial cell.

In some aspects provided herein is a method of treating a patient suffering from a vascular condition or disease. In some embodiments, the method comprises administering a composition comprising a therapeutically effective amount of a population of isolated, engineered PD-1 engager endothelial cells.

In some embodiments, the method comprises administering a composition comprising a therapeutically effective amount of a population of any one of the isolated, engineered PD-1 engager endothelial cells described herein. In some embodiments, the composition further comprises a therapeutically effective carrier. In some embodiments, the administration comprises implantation into the patient's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the vascular condition or disease is selected from the group consisting of vascular injury, cardiovascular disease, vascular disease, ischemic disease, myocardial infarction, congestive heart failure, hypertension, ischemic tissue injury, limb ischemia, stroke, neuropathy, and cerebrovascular disease.

In some aspects, provided herein is a method of producing a population of PD-1 engager endothelial cells from a population of iPSC, ESC, HIP, iPSCO, ESCO, HIPO, iPSCO−, ESCO−, or HIPO− PD-1 engager cells by in vitro differentiation, wherein endogenous β-2 microglobulin (B2M) gene activity and endogenous class II transactivator (CIITA) gene activity have been eliminated and a PD-1 engager molecule is provided on the cell surface. The method comprises: (a) culturing the cells in a first culture medium comprising a GSK inhibitor; (b) culturing the population of cells in a second culture medium comprising VEGF and bFGF to produce a population of pre-endothelial cells; and (c) culturing the population of pre-endothelial cells in a third culture medium comprising a ROCK inhibitor and an ALK inhibitor to produce a population of hypoimmune endothelial cells.

In some embodiments, the GSK inhibitor is CHIR-99021, a derivative thereof, or a variant thereof. In some instances, the GSK inhibitor is at a concentration ranging from about 1 μM to about 10 μM. In some embodiments, the ROCK inhibitor is Y-27632, a derivative thereof, or a variant thereof. In some instances, the ROCK inhibitor is at a concentration ranging from about 1 μM to about 20 μM. In some embodiments, the ALK inhibitor is SB-431542, a derivative thereof, or a variant thereof. In some instances, the ALK inhibitor is at a concentration ranging from about 0.5 μM to about 10 μM.

In some embodiments, the first culture medium comprises from 2 μM to about 10 μM of CHIR-99021. In some embodiments, the second culture medium comprises 50 ng/ml VEGF and 10 ng/ml bFGF. In other embodiments, the second culture medium further comprises Y-27632 and SB-431542. In various embodiments, the third culture medium comprises 10 μM Y-27632 and 1 μM SB-431542. In certain embodiments, the third culture medium further comprises VEGF and bFGF. In particular instances, the first culture medium and/or the second medium is absent of insulin.

In some aspects, provided herein is an isolated, engineered PD-1 engager dopaminergic neuron (DN) differentiated from PD-1 engager cell, wherein endogenous β-2 microglobulin (B2M) gene activity and endogenous class II transactivator (CIITA) gene activity have been eliminated, a PD-1 engager molecule is provided on the cell surface, and the neuron is blood type O and Rh−.

In some embodiments, the isolated PD-1 engager dopaminergic neuron is selected from the group consisting of a neuronal stem cell, neuronal progenitor cell, immature dopaminergic neuron, and mature dopaminergic neuron.

In some aspects, provided herein is a method of treating a patient suffering from a neurodegenerative disease or condition. In some embodiments, the method comprises administering a composition comprising a therapeutically effective amount of a population of any one of the isolated PD-1 engager dopaminergic neurons. In some embodiments, the composition further comprises a therapeutically effective carrier. In some embodiments, the population of the isolated hypoimmune dopaminergic neurons is on a biodegradable scaffold. In some embodiments, the administration may comprise transplantation or injection. In some embodiments, the neurodegenerative disease or condition is selected from the group consisting of Parkinson's disease, Huntington disease, and multiple sclerosis.

In some aspects, provided herein is a method of producing a population of PD-1 engager dopaminergic neurons from a population of PD-1 engager cells by in vitro differentiation, wherein endogenous β-2 microglobulin (B2M) gene activity and endogenous class II transactivator (CIITA) gene activity have been eliminated, a PD-1 engager molecule is provided on the cell surface, the blood group is O and Rh−. In some embodiments, the method comprises (a) culturing the population of cells in a first culture medium comprising one or more factors selected from the group consisting of sonic hedgehog (SHH), BDNF, EGF, bFGF, FGF8, WNT1, retinoic acid, a GSK30 inhibitor, an ALK inhibitor, and a ROCK inhibitor to produce a population of immature dopaminergic neurons; and (b) culturing the population of immature dopaminergic neurons in a second culture medium that is different than the first culture medium to produce a population of dopaminergic neurons.

In some embodiments, the GSKβ inhibitor is CHIR-99021, a derivative thereof, or a variant thereof. In some instances, the GSKβ inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method also comprises isolating the population of hypoimmune dopaminergic neurons from non-dopaminergic neurons. In some embodiments, the method further comprises cryopreserving the isolated population of hypoimmune dopaminergic neurons.

In some aspects, provided herein is an isolated PD-1 engager hypoimmune pancreatic islet cell differentiated from a PD-1 engager cell, wherein endogenous β-2 microglobulin (B2M) gene activity and endogenous class II transactivator (CIITA) gene activity have been eliminated, a PD-1 engager molecule is provided on the cell surface, the blood type is O and Rh−.

In some embodiments, the isolated PD-1 engager pancreatic islet cell is selected from the group consisting of a pancreatic islet progenitor cell, immature pancreatic islet cell, and mature pancreatic islet cell.

In some aspects, provided herein is a method of treating a patient suffering from diabetes. The method comprises administering a composition comprising a therapeutically effective amount of a population of any one of the isolated PD-1 engager pancreatic islet cells described herein. In some embodiments, the composition further comprises a therapeutically effective carrier. In some embodiments, the population of the isolated hypoimmune pancreatic islet cells is on a biodegradable scaffold. In some instances, the administration comprises transplantation or injection.

In some aspects, provided herein is a method of producing a population of PD-1 engager pancreatic islet cells from a population of HIPO− cells by in vitro differentiation, wherein endogenous 3-2 microglobulin (B2M) gene activity and endogenous class II transactivator (CIITA) gene activity have been eliminated, a PD-1 engager molecule is provided on the cell surface, the blood type is O and Rh− in the HIPO− cells. The method comprises: (a) culturing the population of PD-1 engager cells in a first culture medium comprising one or more factors selected from the group consisting insulin-like growth factor (IGF), transforming growth factor (TGF), fibroblast growth factor (EGF), epidermal growth factor (EGF), hepatocyte growth factor (HGF), sonic hedgehog (SHH), and vascular endothelial growth factor (VEGF), transforming growth factor-β (TGFβ) superfamily, bone morphogenic protein-2 (BMP2), bone morphogenic protein-7 (BMP7), a GSK30 inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of hypoimmune pancreatic islet cells.

In some embodiments, the GSK inhibitor is CHIR-99021, a derivative thereof, or a variant thereof. In some instances, the GSK inhibitor is at a concentration ranging from about 2 μM to about 10 μM. In some embodiments, the ALK inhibitor is SB-431542, a derivative thereof, or a variant thereof. In some instances, the ALK inhibitor is at a concentration ranging from about 1 μM to about 10 μM. In some embodiments, the first culture medium and/or second culture medium are absent of animal serum.

In some embodiments, the method also comprises isolating the population of PD-1 engager pancreatic islet cells from non-pancreatic islet cells. In some embodiments, the method further comprises cryopreserving the isolated population of hypoimmune pancreatic islet cells.

In some aspects, provided herein is an isolated, engineered PD-1 engager retinal pigmented epithelium (RPE) cell differentiated from a PD-1 engager cell, wherein endogenous β-2 microglobulin (B2M) gene activity and endogenous class II transactivator (CIITA) gene activity have been eliminated, a PD-1 engager molecule is provided on the cell surface, the blood type is O and Rh−.

In some embodiments, the isolated PD-1 engager cell RPE cell is selected from the group consisting of an RPE progenitor cell, immature RPE cell, mature RPE cell, and functional RPE cell.

In some aspects, provided herein is a method of treating a patient suffering from an ocular condition. The method comprises administering a composition comprising a therapeutically effective amount of a population of any one of a population of the isolated PD-1 engager cell RPE cells described herein. In some embodiments, the composition further comprises a therapeutically effective carrier. In some embodiments, the population of the isolated hypoimmune RPE cells is on a biodegradable scaffold. In some embodiments, the administration comprises transplantation or injection to the patient's retina. In some embodiments, the ocular condition is selected from the group consisting of wet macular degeneration, dry macular degeneration, juvenile macular degeneration, Leber's Congenital Ameurosis, retinitis pigmentosa, and retinal detachment.

In some aspects, provided herein is a method of producing a population of PD-1 engager retinal pigmented epithelium (RPE) cells from a population of PD-1 engager cells by in vitro differentiation, wherein endogenous β-2 microglobulin (B2M) gene activity and endogenous class II transactivator (CIITA) gene activity have been eliminated and a PD-1 engager molecule is provided on the cell surface. The method comprises: (a) culturing the population of PD-1 engager cells in a first culture medium comprising any one of the factors selected from the group consisting of activin A, bFGF, BMP4/7, DKK1, IGF1, noggin, a BMP inhibitor, an ALK inhibitor, a ROCK inhibitor, and a VEGFR inhibitor to produce a population of pre-RPE cells; and (b) culturing the population of pre-RPE cells in a second culture medium that is different than the first culture medium to produce a population of hypoimmune RPE cells.

In some embodiments, the ALK inhibitor is SB-431542, a derivative thereof, or a variant thereof. In some instances, the ALK inhibitor is at a concentration ranging from about 2 μM to about 10 μM. In some embodiments, the ROCK inhibitor is Y-27632, a derivative thereof, or a variant thereof. In some instances, the ROCK inhibitor is at a concentration ranging from about 1 μM to about 10 μM.

In some embodiments, the first culture medium and/or second culture medium are absent of animal serum.

In some embodiments, the method further comprises isolating the population of PD-1 engager RPE cells from non-RPE cells. In some embodiments, the method further comprises cryopreserving the isolated population of hypoimmune RPE cells.

### E. Transplantation of HI PD-1 Engager Cells

As will be appreciated by those in the art that the HI PD-1 engager cells cells are transplated using techniques known in the art. In general, the HI PD-1 engager cells of the invention are transplanted either intravenously or by injection at particular locations in the patient. When transplanted at particular locations, the cells may be suspended in a gel matrix to prevent dispersion while they take hold.

In order that the invention described herein may be more fully understood, the following examples are set forth. It should be understood that these examples are for illustrative purposes only and are not to be construed as limiting this invention in any manner.

### VIII. EXAMPLES

### Example 1: A PD-1 Engager Expressed on Engineered Cells Inhibits NK Cell Killing

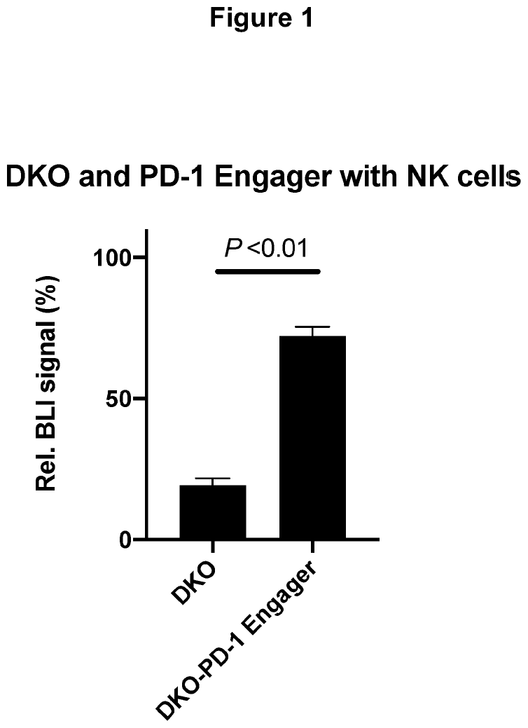

B2M−/− CIITA−/− iPSC-derived endothelial cells (DKO iECs) underwent rapid killing by IL-2 activated NK cells. The PD-1 engager was expressed on B2M−/− CIITA−/− iECs using lentiviral transduction. The killing of B2M−/− CIITA−/− PD-1 engager transgenic iECs (DKO-PD-1 Engager) was significantly mitigated as shown by a bioluminescence imaging (BLI) killing assay. (FIG. 1.)

### Example 2: A PD-1 Engager Expressed on Engineered Cells Inhibits CD8 T Cell Killing

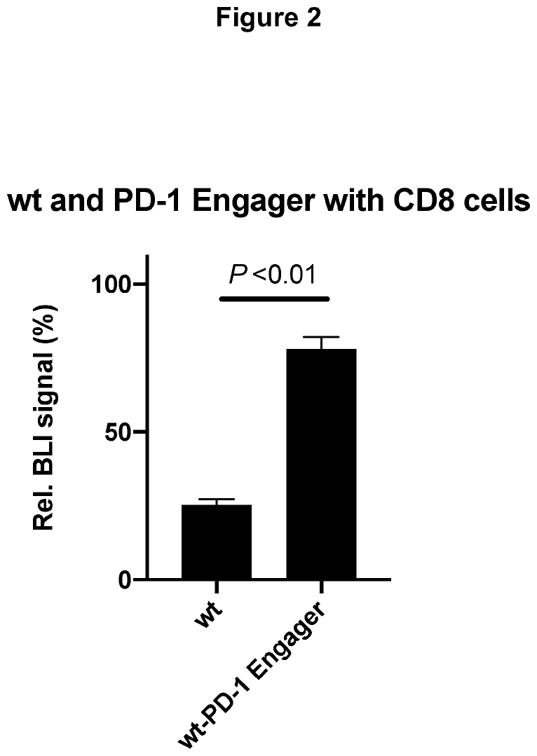

PBMCs from an allogeneic healthy donor who is HLA-A2 negative were incubated with HLA-A2-positive wild type (wt) iPSC-derived endothelial cells (iECs) for 14 days. Cells were harvested and CD3+CD8+ T cells were sorted using flow cytometry. Wt iECs underwent T cell killing. The PD-1 engager was expressed on wt iECs using lentiviral transduction. The killing of wt PD-1 engager transgenic iECs was significantly mitigated as shown by a BLI killing assay. (FIG. 2.)

### Example 3: A PD-1 Engager Expressed on Engineered Human Pancreatic Beta Cells Inhibits CD8 T Cell Killing

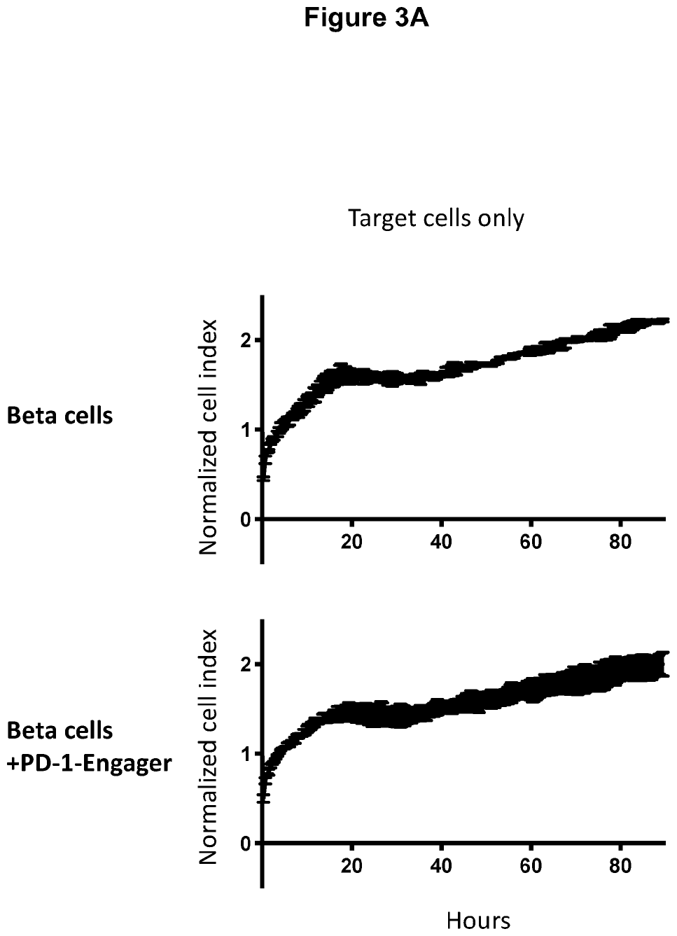

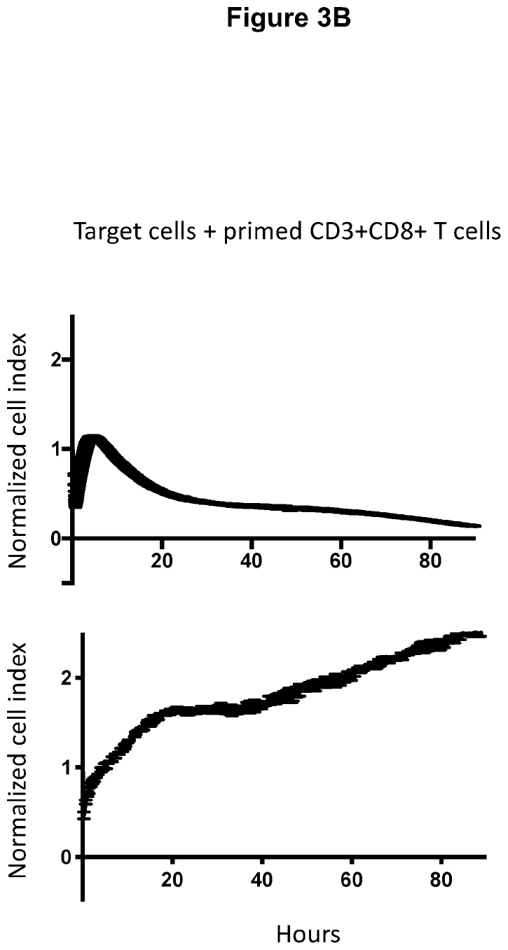

PBMCs from an allogeneic healthy donor who is HLA-A2 negative were incubated with HLA-A2-positive wild type (wt) iPSC-derived endothelial cells (iECs) for 14 days. Cells were harvested and CD3+CD8+ T cells were sorted using flow cytometry. Human pancreatic beta cells that were HLA-A2 positive were grown on in vitro impedance assay E-plates. In the upper row, the beta cells were not modified. In the bottom row, the beta cells expressed the PD-1 engager. The PD-1 engager was expressed using lentiviral transduction. A PD-1 Engager sequence (SEQ ID NO:24) with CD8a signal peptide (SEQ ID NO:25) and myc-tag (SEQ ID NO:26) for detection in flow cytometry was used. The unmodified beta cells underwent T cell killing. The PD-1 engager transgenic beta cells survived. (FIGS. 3A and 3B)

### Example 4: The PD-1 Engager Cells Bind Recombinant PD-1

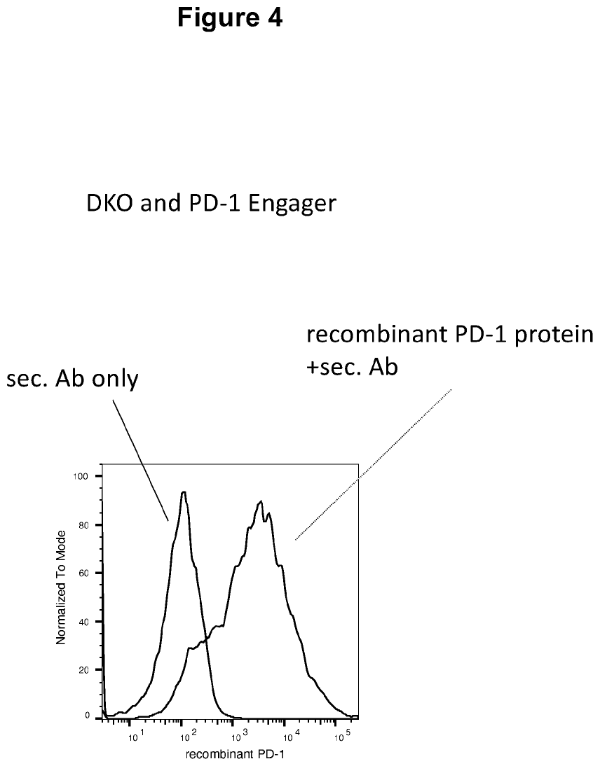

B2M−/− CIITA−/− PD-I engager transgenic iECs (DKO-PD-1 Engager) were incubated with a recombinant human PD-1 and a secondary antibody. The flow cytometry histogram shows specific binding of the recombinant human PD-1 to the DKO-PD-1 Engager cells. (FIG. 4)

### Example 5: Generation of a TIM3 Engager Cell

Lentivirus preparation. A TIM3 engager (SEQ ID NO:27) was expressed under the enhanced EF1α promoter. It comprised a CD8a signal peptide (SEQ ID NO:28), a myc tag (SEQ ID NO:26), an anti-TIM3 antibody heavy chain variable region (SEQ ID NO:29), a (GGGG)₃ linker (SEQ ID NO:30), an anti-TIM3 antibody light chain variable region (SEQ ID NO:31), a CD8a Hinge (SEQ ID NO:3), and a platelet derived growth factor receptor (PDGFR) transmembrane domain (SEQ ID NO:4). An RFP-Blasticidin dual fusion marker was expressed under an RSV promoter.

The expression lentivector was co-transfected with lentiviral packaging plasmids (Cat #: HT-pack, Gentarget, San Diego) into the lentivirus production cell line (Cat #: TLV-C, Gentarget). The lentivirus was packaged in DMEM medium with 10% serum following GenTarget's virus production protocols. The lentivirus was then concentrated to obtain the desired virus titer. Virus titers were measured via an ELISA P24 assay according to the kit production.

Lentiviral transduction of the TIM3 engager. In a pre-coated 12-well plate, human induced pluripotent stem cell (iPSC)-derived endothelial cells (iECs) with a double knockout of B2M and CIITA (iECs DKO) were transduced with the lentivirus described above. They were plated at a density of 5×10⁴ and then incubated overnight at 37° C. at 5% CO₂. The next day, cells were incubated overnight at 37° C., 5% CO₂ with lentiviral particles carrying a transgene for the TIM3 engager under the enhanced EF1α promoter (Gentarget, custom product). The lentiviruses further contained an RFP selection marker and were used at a multiplicity of infection of 4. Polybrene (8 μg/m, Millipore) was added to the media and the plate was centrifuged at 800 g for 30 min prior to the overnight incubation. Cell populations were sorted on FACSAria (BD Biosciences) using the RFP tag.

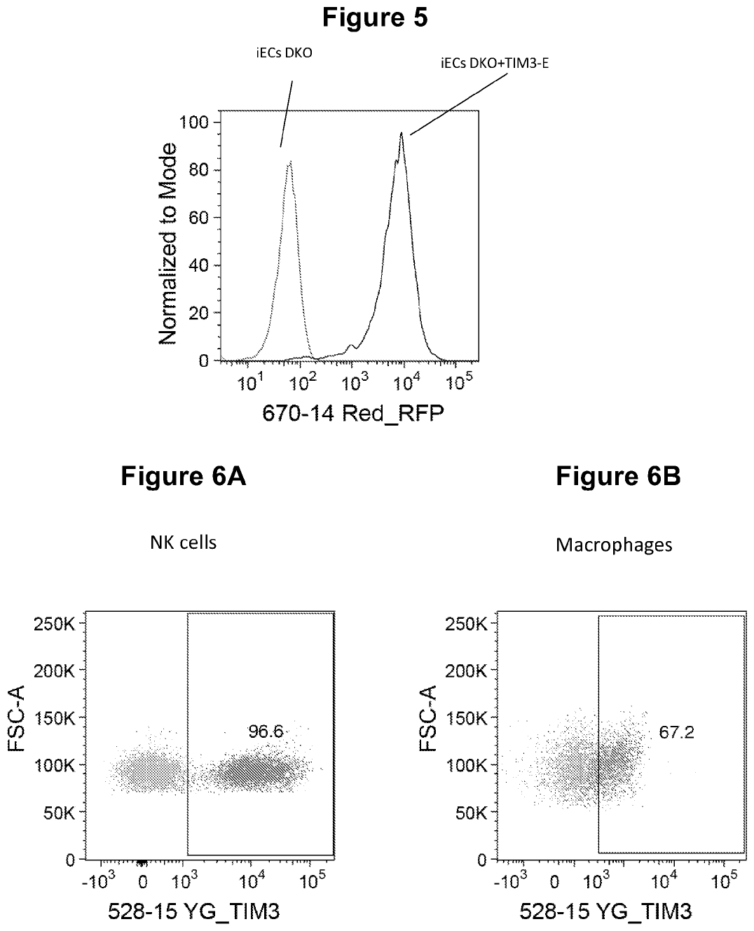

Flow cytometry analysis showing that the TIM3 Engager protein is expression in transduced DKO induced embryonic stem cells (iEC). The TIM3 Engager transgene linked to the red fluorescent protein (RFP) tag was quantified by flow cytometry. The histogram shows the RFP signal of untransduced iECs DKO (left peak) and transduced iECs DKO+TIM3-E (right peak), thus confirming their expression of the TIM3 Engager. (FIG. 5.)

### Example 6: TIM3 Engagement Protects Cells from Immune Cell Killing

Flow cytometry analysis for TIM3 surface expression on primary human peripheral blood NK cells and macrophages showed that TIM3 engagement protected the cells from NK and macrophage cells.

FIG. 6A shows that 96.6% of NK cells were positive for TIM3 and FIG. 3B shows that 67.2% of macrophages were positive for TIM3 (FIG. 6B).

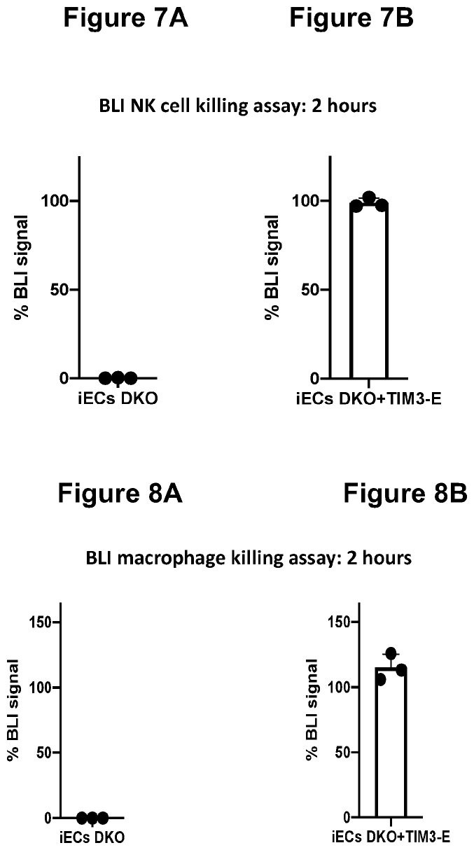

A BLI killing assay was examined over 2 hours with NK cells. iECs DKO expressing firefly luciferase or iECs DKO+TIM3 engager (TIM3-E) expressing firefly luciferase were plated and allowed to attach for 16 hours. Then, human peripheral blood NK cells were added to the wells and incubated for 2 hours. The percent BLI signal after the incubation is shown. The BLI signal for iECs DKO completely vanished (FIG. 7A). The BLI signal of iECs DKO+TIM3-E remained steady over the 2 hours (FIG. 7B).

FIGS. 8A and 8B: BLI killing assay over 2 hours with macrophages. Human iECs DKO or iECs DKO+TIM3-E expressing firefly luciferase were plated and allowed to attach for 16 hours. Then, human peripheral blood macrophages were added to the wells and incubated for 2 hours. The percent BLI signal after the incubation is shown. The BLI signal for iECs DKO completely vanished (FIG. 8A). The BLI signal of iECs DKO+TIM3-E remained steady over the 2 hours (FIG. 8B).

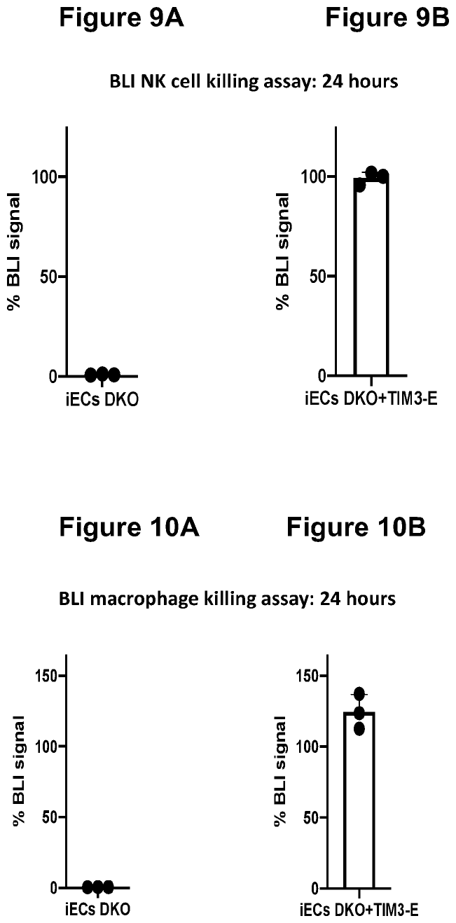

FIGS. 9A and 9B: BLI killing assay over 24 hours with NK cells. Firefly luciferase-expressing human iECs DKO or iECs DKO+TIM3-E were plated and allowed to attach for 16 hours. Then, human peripheral blood NK cells were added to the wells and incubated for 24 hours. The percent BLI signal after the 24 hours NK incubation is shown. The BLI signal for iECs DKO completely vanished (FIG. 9A). The BLI signal of iECs DKO+TIM3-E remained steady over the 24 hours (FIG. 9B).

FIGS. 10A and 10B: BLI killing assay over 24 hours with macrophages. Firefly luciferase-expressing human iECs DKO and iECs DKO+TIM3-E were plated and allowed to attach for 16 hours. Then, human peripheral blood macrophages were added to the wells and incubated for 24 hours. The percent BLI signal after the 24 hours macrophage incubation is shown. The BLI signal for iECs DKO completely vanished (FIG. 10A). The BLI signal of iECs DKO+TIM3-E remained steady over the 24 hours and even increased somewhat (FIG. 10B).

### Example 7: Generation of a LILRB3 Engager Cell

Lentivirus preparation. The LILRB3 engager (SEQ ID NO:32) was expressed under the enhanced EF1α promoter. It comprised a CD8a signal peptide (SEQ ID NO:28), a myc tag (SEQ ID NO:26), an anti-LILRB3 antibody heavy chain variable region (SEQ ID NO:33), a (GGGG)₃ linker (SEQ ID NO:30), an anti-LILRB3 antibody light chain variable region (SEQ ID NO:34), a CD8a Hinge (SEQ ID NO:3), and a platelet derived growth factor receptor (PDGFR) transmembrane domain (SEQ ID NO:4). An RFP-Blasticidin dual fusion marker was expressed under an RSV promoter.

The expression lentivector was co-transfected with lentiviral packaging plasmids (Cat #: HT-pack, Gentarget, San Diego) into the lentivirus production cell line (Cat #: TLV-C, Gentarget). The lentivirus was packaged in DMEM medium with 10% serum following GenTarget's virus production protocols. The lentivirus was then concentrated to obtain the desired virus titer. Virus titers were measured via ELISA P24 assay according to the kit production.

Lentiviral transduction of the LILRB3 engager. In a pre-coated 12-well plate, human induced pluripotent stem cell (iPSC)-derived endothelial cells (iECs) with a double knockout of B2M and CIITA (iECs DKO) were transduced with the lentivirus described above. They were plated at a density of 5×10⁴ and then incubated overnight at 37° C. at 5% CO₂. The next day, cells were incubated overnight at 37° C., 5% CO₂ with lentiviral particles carrying a transgene for the LILRB3 engager under the enhanced EF1α promoter (Gentarget). The lentiviruses further contained an RFP selection marker and were used at a multiplicity of infection of 4. Polybrene (8 μg/m, Millipore) was added to the media and the plate was centrifuged at 800 g for 30 min prior to the overnight incubation. Cell populations were sorted on FACSAria (BD Biosciences) using the RFP tag.

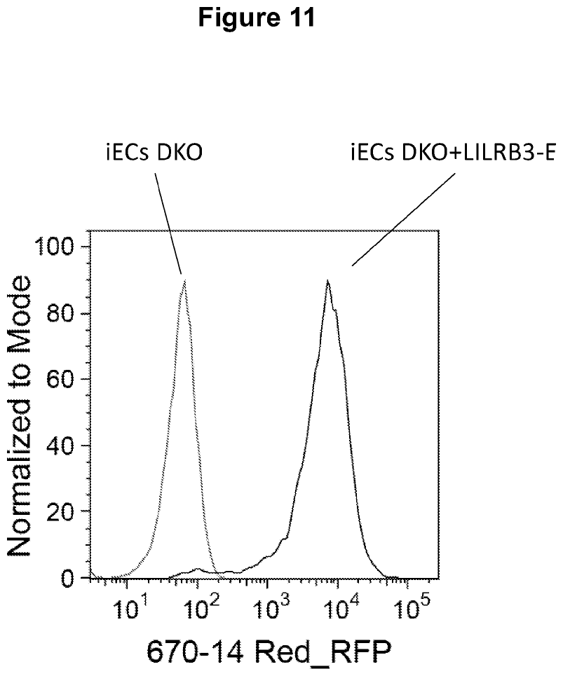

Flow cytometry analysis showing that the LILRB3 Engager protein is expression in transduced DKO induced embryonic stem cells (iEC). The LILRB3 Engager transgene linked to the red fluorescent protein (RFP) tag was quantified by flow cytometry. The histogram shows the RFP signal of untransduced iECs DKO (left peak) and transduced iECs DKO+LILRB3-E (right peak), thus confirming their expression of the LILRB3 Engager (FIG. 11.)

### Example 8: LILRB3 Engagement Protects Cells from Immune Cell Killing

Flow cytometry analysis for LILRB3 surface expression on primary human peripheral blood NK cells and macrophages showed that LILRB3 is expressed on some NK cells and most macrophages.

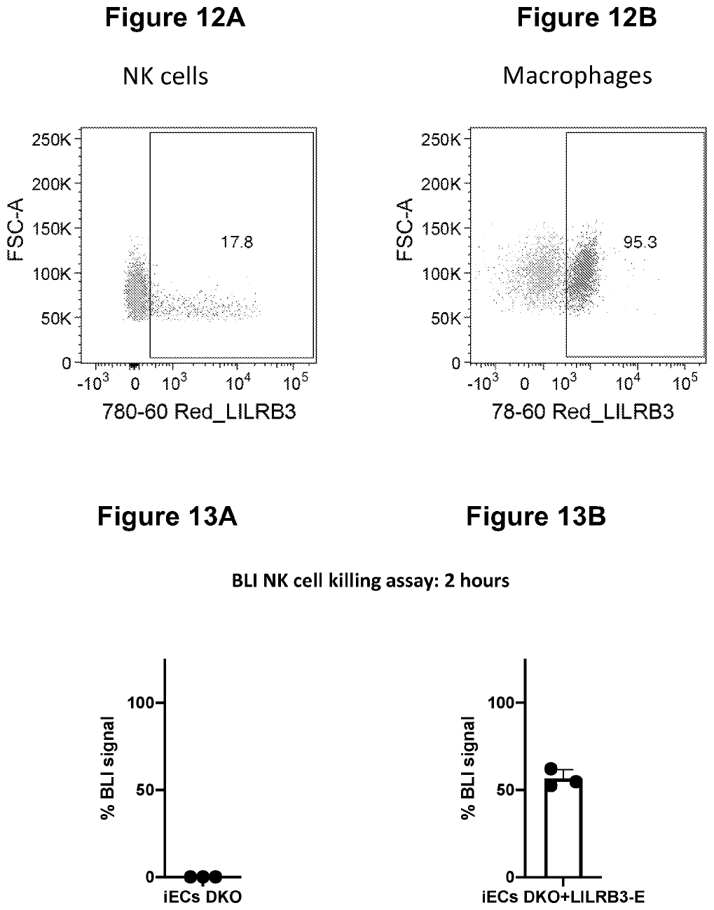

FIG. 12A shows that 17.8% of NK cells were positive for LILRB3 and FIG. 12B shows that 95.36% of macrophages were positive for LILRB3.

A BLI killing assay was examined over 2 hours with NK cells. iECs DKO expressing firefly luciferase or iECs DKO+LILRB3-E expressing firefly luciferase were plated and allowed to attach for 16 hours. Then, human peripheral blood NK cells were added to the wells and incubated for 2 hours. The percent BLI signal after the incubation is shown. The BLI signal for iECs DKO completely vanished (FIG. 13A). The BLI signal of iECs DKO+LILRB3-E dropped markedly less over the 2 hours (FIG. 13B).

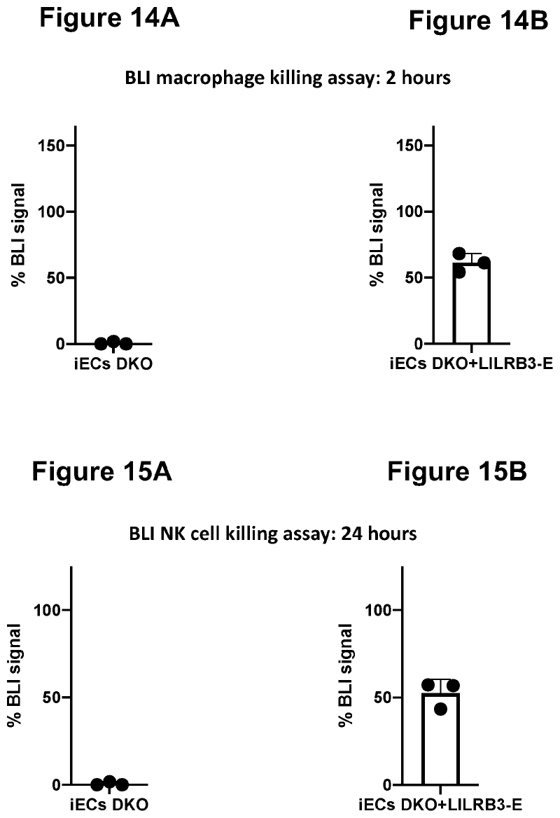

FIGS. 14A and 13B: BLI killing assay over 2 hours with macrophages. Human iECs DKO or iECs DKO+LILRB3-E expressing firefly luciferase were plated and allowed to attach for 16 hours. Then, human peripheral blood macrophages were added to the wells and incubated for 2 hours. The percent BLI signal after the incubation is shown. The BLI signal for iECs DKO completely vanished (FIG. 14A). The BLI signal of iECs DKO+LILRB3-E dropped markedly less over the 2 hours (FIG. 4B).

FIGS. 15A and 15B: BLI killing assay over 24 hours with NK cells. Firefly luciferase-expressing human iECs DKO or iECs DKO+LILRB3-E were plated and allowed to attach for 16 hours. Then, human peripheral blood NK cells were added to the wells and incubated for 24 hours. The percent BLI signal after the 24 hours NK incubation is shown. The BLI signal for iECs DKO completely vanished (FIG. 15A). The BLI signal of iECs DKO+LILRB3-E dropped markedly less over the 24 hours (FIG. 15B).

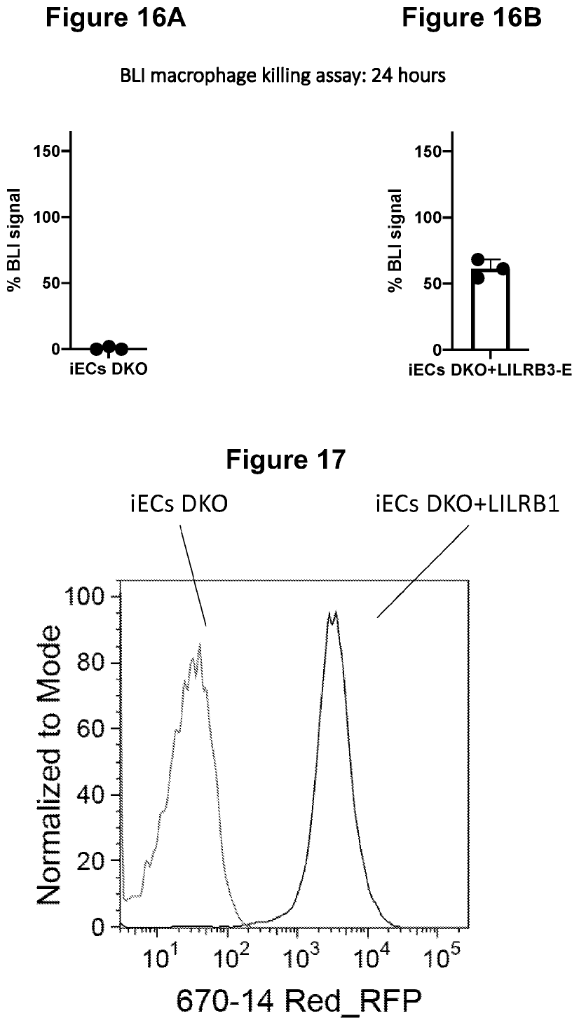

FIGS. 16A and 16B: BLI killing assay over 24 hours with macrophages. Firefly luciferase-expressing human iECs DKO and iECs DKO+LILRB3-E were plated and allowed to attach for 16 hours. Then, human peripheral blood macrophages were added to the wells and incubated for 24 hours. The percent BLI signal after the 24 hours macrophage incubation is shown. The BLI signal for iECs DKO completely vanished (FIG. 16A). The BLI signal of iECs DKO+LILRB3-E dropped markedly less over the 24 hours and even increased somewhat (FIG. 16B).

### Example 9: Generation of a LILRB1 Engager Cell

Lentivirus preparation. The LILRB1 engager (SEQ ID NO:35) was expressed under the enhanced EF1α promoter and an RFP-Blasticidin dual fusion marker was expressed under an RSV promoter. It comprised a CD8a signal peptide (SEQ ID NO:28), a myc tag (SEQ ID NO:26), an anti-LILRB1 antibody heavy chain variable region (SEQ ID NO:36), a (GGGG)₃ linker (SEQ ID NO:30), an anti-LILRB1 antibody light chain variable region (SEQ ID NO:37), a CD8a Hinge (SEQ ID NO:3), and a platelet derived growth factor receptor (PDGFR) transmembrane domain (SEQ ID NO:4). An RFP-Blasticidin dual fusion marker was expressed under an RSV promoter.

The expression lentivector was co-transfected with lentiviral packaging plasmids (Cat #: HT-pack, Gentarget, San Diego) into the lentivirus production cell line (Cat #: TLV-C, Gentarget). The lentivirus was packaged in DMEM medium with 10% serum following GenTarget's virus production protocols. The lentivirus was then concentrated to obtain the desired virus titer. Virus titers were measured via ELISA P24 assay according to the kit production.

Lentiviral transduction of the LILRB1 engager. In a pre-coated 12-well plate, human induced pluripotent stem cell (iPSC)-derived endothelial cells (iECs) with a double knockout of B2M and CIITA (iECs DKO) were transduced with the lentivirus described above. They were plated at a density of 5×10⁴ and then incubated overnight at 37° C. at 5% CO₂. The next day, cells were incubated overnight at 37° C., 5% CO₂ with lentiviral particles carrying a transgene for the LILRB1 engager under the enhanced EF1a promoter (Gentarget). The lentiviruses further contained an RFP selection marker and were used at a multiplicity of infection of 4. Polybrene (8 μg/m, Millipore) was added to the media and the plate was centrifuged at 800 g for 30 min prior to the overnight incubation. Cell populations were sorted on FACSAria (BD Biosciences) using the RFP tag.

Flow cytometry analysis showing that the LILRB1 Engager protein is expression in transduced DKO induced embryonic stem cells (iEC). The LILRB1 Engager transgene linked to the red fluorescent protein (RFP) tag was quantified by flow cytometry. The histogram shows the RFP signal of untransduced iECs DKO (left peak) and transduced iECs DKO+LILRB1-E (right peak), thus confirming their expression of the LILRB1 Engager (FIG. 17.)

### Example 10: LILRB1 Engagement Protects Cells from Immune Cell Killing

Flow cytometry analysis for LILRB1 surface expression on primary human peripheral blood NK cells and macrophages showed that LILRB1 is expressed on many NK cells and most macrophages.

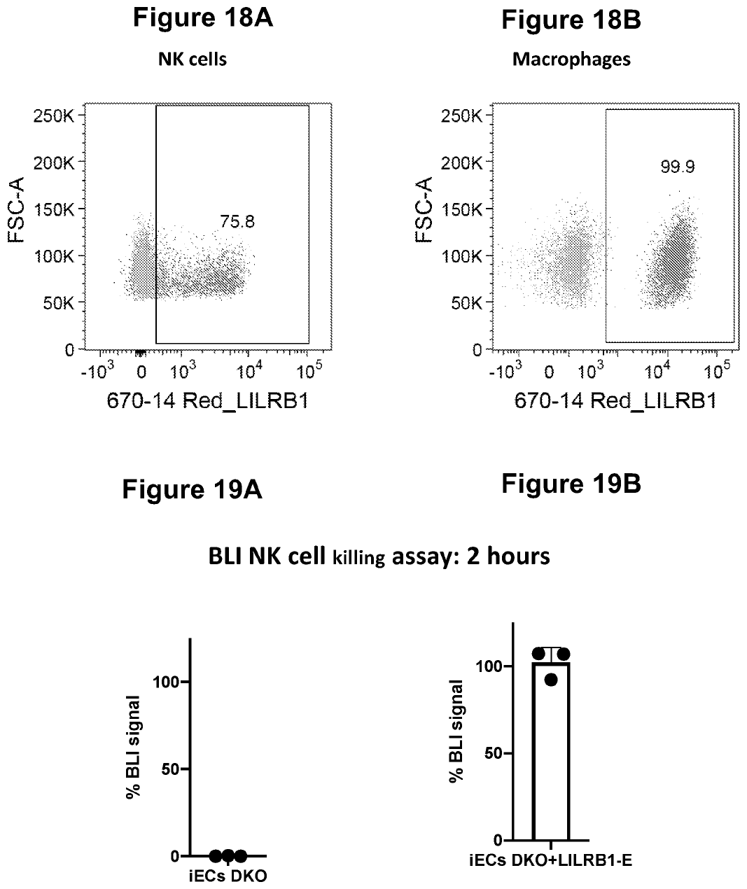

FIG. 18A shows that 75.8% of NK cells were positive for LILRB1 and FIG. 18B shows that 99.96% of macrophages were positive for LILRB1.

A BLI killing assay was examined over 2 hours with NK cells. iECs DKO expressing firefly luciferase or iECs DKO+LILRB1-E expressing firefly luciferase were plated and allowed to attach for 16 hours. Then, human peripheral blood NK cells were added to the wells and incubated for 2 hours. The percent BLI signal after the incubation is shown. The BLI signal for iECs DKO completely vanished (FIG. 19A). The BLI signal of iECs DKO+LILRB1-E remained steady over the 2 hours (FIG. 19B).

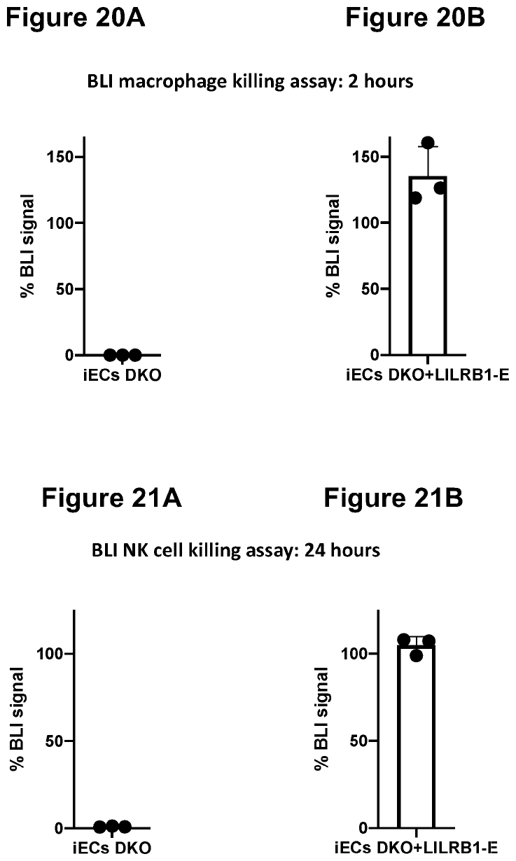

FIGS. 20A and 20B: BLI killing assay over 2 hours with macrophages. Human iECs DKO or iECs DKO+LILRB1-E expressing firefly luciferase were plated and allowed to attach for 16 hours. Then, human peripheral blood macrophages were added to the wells and incubated for 2 hours. The percent BLI signal after the incubation is shown. The BLI signal for iECs DKO completely vanished (FIG. 20A). The BLI signal of iECs DKO+LILRB1-E remained steady over the 2 hours (FIG. 20B).

FIGS. 21A and 21B: BLI killing assay over 24 hours with NK cells. Firefly luciferase-expressing human iECs DKO or iECs DKO+LILRB1-E were plated and allowed to attach for 16 hours. Then, human peripheral blood NK cells were added to the wells and incubated for 24 hours. The percent BLI signal after the 24 hours NK incubation is shown. The BLI signal for iECs DKO completely vanished (FIG. 21A). The BLI signal of iECs DKO+LILRB1-E remained steady over the 24 hours (FIG. 21B).

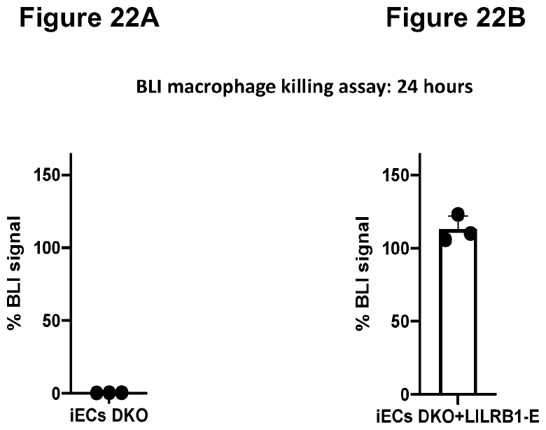

FIGS. 22A and 22B: BLI killing assay over 24 hours with macrophages. Firefly luciferase-expressing human iECs DKO and iECs DKO+LILRB1-E were plated and allowed to attach for 16 hours. Then, human peripheral blood macrophages were added to the wells and incubated for 24 hours. The percent BLI signal after the 24 hours macrophage incubation is shown. The BLI signal for iECs DKO completely vanished (FIG. 22A). The BLI signal of iECs DKO+LILRB1-E remained steady over the 24 hours and even increased somewhat (FIG. 22B).

### Example 11: Immune Checkpoint Engagers are Stably Expressed after Differentiation

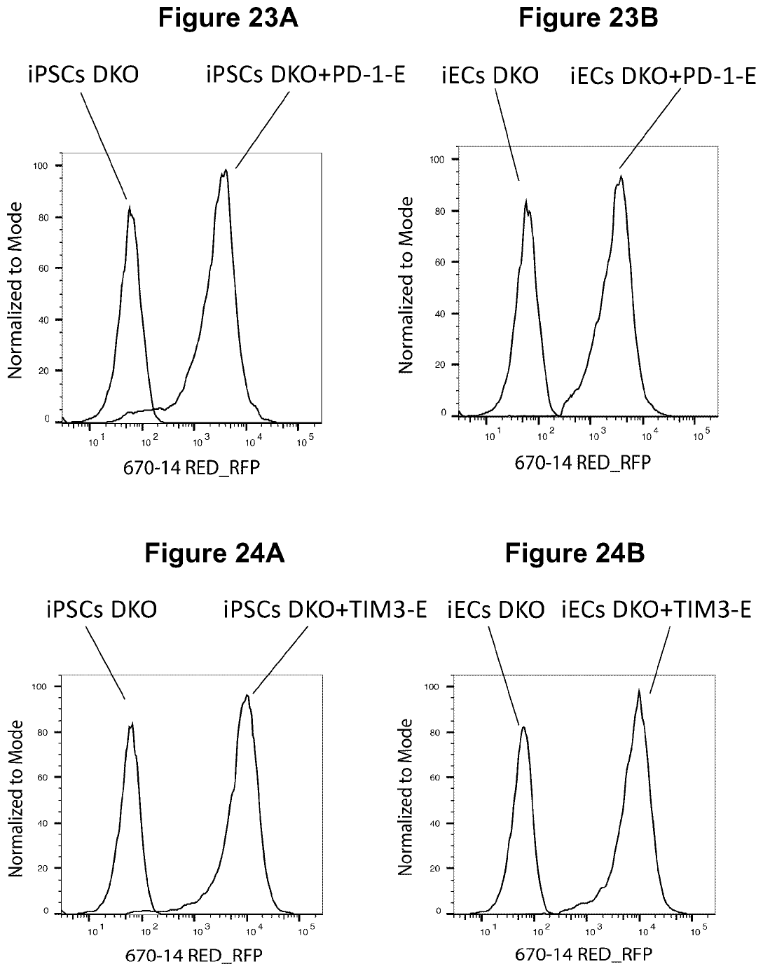

Expression of the PD-1 Engager transgene is maintained during differentiation. iPSCs DKO were transduced to express the PD-1 Engager using lentiviral particles (iPSCs DKO+PD-1-E). Flow cytometry shows that robust expression can be achieved (FIG. 23A). When iPSCs DKO+PD-1-E were differentiated into iECs DKO+PD-1-E, transgene expression remained stable (FIG. 23B).

Expression of the TIM3 Engager transgene is maintained during differentiation. iPSCs DKO were transduced to express the TIM3 Engager using lentiviral particles (iPSCs DKO+TIM3-E). Flow cytometry shows that robust expression can be achieved (FIG. 24A). When iPSCs DKO+TIM3-E were differentiated into iECs DKO+TIM3-E, transgene expression remained stable (FIG. 24B).

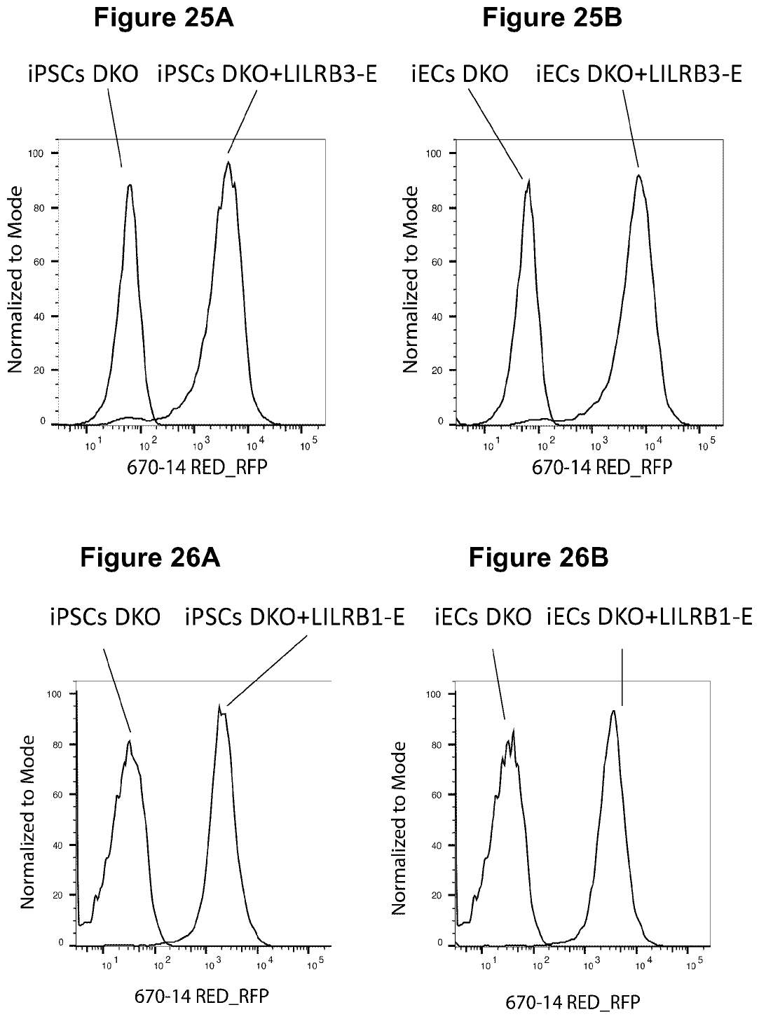

Expression of the LILRB3 Engager transgene is maintained during differentiation. iPSCs DKO were transduced to express the LILRB3 Engager using lentiviral particles (iPSCs DKO+LILRB3-E). Flow cytometry shows that robust expression can be achieved (FIG. 25A). When iPSCs DKO+LILRB3-E were differentiated into iECs DKO+LILRB3-E, transgene expression remained stable (FIG. 25B).

Expression of the LILRB1 Engager transgene is maintained during differentiation. iPSCs DKO were transduced to express the LILRB1 Engager using lentiviral particles (iPSCs DKO+LILRB1-E). Flow cytometry shows that robust expression can be achieved (FIG. 26A). When iPSCs DKO+LILRB1-E were differentiated into iECs DKO+LILRB1-E, transgene expression remained stable (FIG. 26B).

### Example 12: Threshold Immune Checkpoint Engager Levels Required for Protection

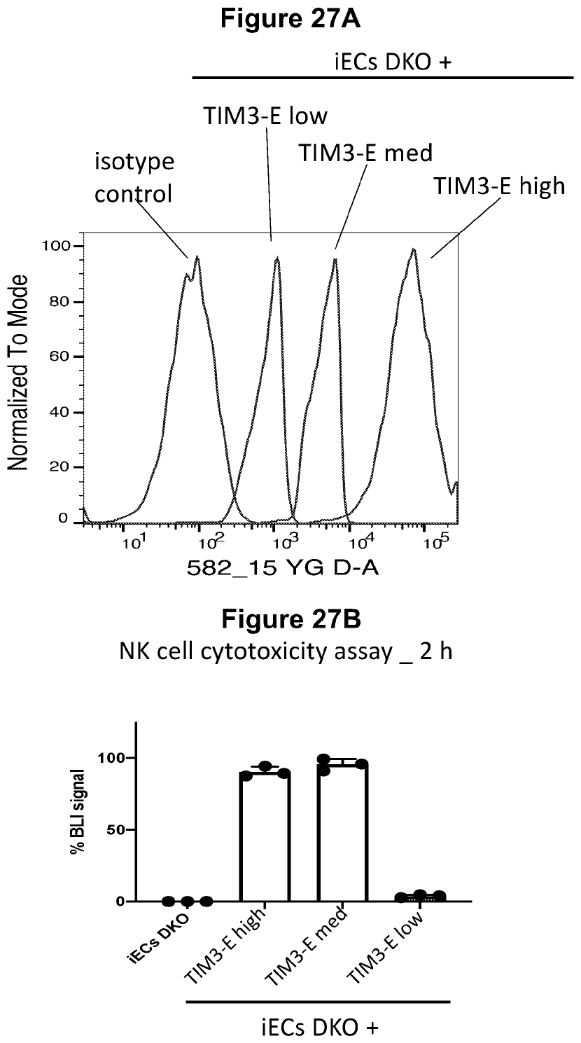

FIGS. 27A and 27B show the threshold for TIM3-E expression required to protect cells from NK cell killing. In order to assess whether there is a functional threshold for the expression of the TIM3-E to exert its protective effect from NK cell killing, iECs DKO were transduced with lentiviral particles carrying the TIM3-E transgene. After 3 days, the transduced pool of iECs was sorted into three populations based on their TIM3-E expression. The three populations are labeled iECs DKO+TIM3-E high, med, and low (FIG. 27A). For the subsequent BLI killing assay, the different populations were incubated with NK cells for 2 hours (FIG. 27B). All target cells were luciferase positive. The percent BLI signal after the 2 hours NK incubation is shown. The BLI signal for iECs DKO completely vanished. The BLI signals for iECs DKO+TIM3-E high and med remained stable, but the BLI signals for iECs DKO+TIM3-E low also dropped to the background. These results show that the functional threshold for the TIM3-E is between the low and med expression levels.

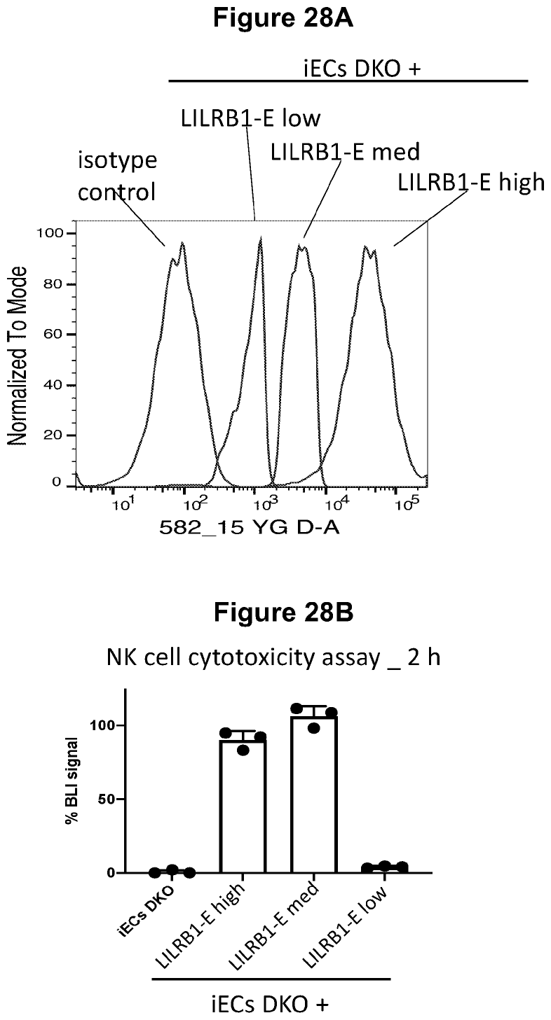

FIGS. 28A and 28B show the threshold for LILRB1-E expression required to protect cells from NK cell killing. In order to assess whether there is a functional threshold for the expression of the LILRB1-E to exert its protective effect from NK cell killing, iECs DKO were transduced with lentiviral particles carrying the LILRB1-E transgene. After 3 days, the transduced pool of iECs was sorted into three populations based on their LILRB1-E expression. The three populations are labeled iECs DKO+LILRB1-E high, med, and low (FIG. 28A). For the subsequent BLI killing assay, the different populations were incubated with NK cells for 2 hours (FIG. 28B). All target cells were luciferase positive. The percent BLI signal after the 2 hours NK incubation is shown. The BLI signal for iECs DKO completely vanished. The BLI signals for iECs DKO+LILRB1-E high and med remained stable, but the BLI signals for iECs DKO+LILRB1-E low also dropped to the background. These results show that the functional threshold for the LILRB1-E is between the low and med expression levels.

### Methods

NK cell culture. Human primary NK cells were purchased from Stemcell Technologies (70036, Vancouver, Canada) and were cultured in RPMI-1640 plus 10% FCS hi and 1% pen/strep before performing the assays.

Human iPSC culture and transduction to express firefly luciferase. Human wt or B2M−/− CIITA−/− iPSCs were cultured on diluted feeder-free matrigel (hESC qualified, BD Biosciences, San Jose, CA)-coated 10 cm dishes in Essential 8 Flex medium (Thermo Fisher Scientific). Medium was changed every 24 h and Versene (Gibco) was used for cell passaging at a ratio of 1:6. For luciferase transduction, 1×10⁵ iPSCs were plated in one 6-well and incubated overnight at 37° C. with 5% CO₂. The next day, medium was changed and one vial of Fluc lentiviral particles expressing luciferase II gene under re-engineered EF1α promotor (Gen Target, San Diego, CA) was added to 1.5 ml medium. After 36 h, 1 ml of cell medium was added. After 24 h, complete medium change was performed. After 2 d, luciferase expression was confirmed by adding D-luciferin (Promega, Madison, WI). Signals were quantified in p/s/cm2/sr.

Differentiation of iPSC-derived endothelial cells (iECs). Fluc+ wt or B2M−/− CIITA−/− iECs were differentiated from FLuc+ B2M−/− CIITA−/− iPSCs as follows. The differentiation protocol was initiated at 60% iPSC confluency, and medium was changed to RPMI-1640 containing 2% B-27 minus insulin (both Gibco, a Thermo Fisher Scientific Brand) and 5 μM CHIR-99021 (Selleckchem, Munich, Germany). On day 2, the medium was changed to reduced medium: RPMI-1640 containing 2% B-27 minus insulin (Gibco) and 2 μM CHIR-99021 (Selleckchem). From culture day 4 to 7, cells were exposed to RPMI-1640 EC medium, RPMI-1640 containing 2% B-27 minus insulin plus 50 ng/ml human vascular endothelial growth factor (VEGF; R&D Systems, Minneapolis, MN), 10 ng/ml human fibroblast growth factor basic (FGFb; R&D Systems), 10 μM Y-27632 (Sigma-Aldrich), and 1 μM SB 431542 (Sigma-Aldrich, St. Louis, MO). Endothelial cell clusters were visible from day 7 and cells were maintained in Endothelial Cell Basal Medium 2 (PromoCell, Heidelberg, Germany) plus supplements, 10% FCS hi (Gibco), 1% pen/strep, 25 ng/ml VEGF, 2 ng/ml FGFb, 10 μM Y-27632 (Sigma-Aldrich), and 1 μM SB 431542 (Sigma-Aldrich). The differentiation protocol was completed after 14 days; undifferentiated cells detached during the differentiation process. TrypLE Express (Gibco) was used for passaging the cells 1:3 every 3 to 4 days.

Macrophage differentiation from PBMCs. PBMCs were isolated by Ficoll separation from fresh blood and re-suspended in RPMI-1640 with 10% FCS HI and 1% penicillin-streptomycin (all Gibco). Cells were plated in 24-well plates at a concentration of 1×10⁶ cells per milliliter. 10 ng ml⁻¹ of human M-CSF (PeproTech) was added to the medium. The medium was changed every other day. From day 6 onward, 1 μg ml⁻¹ of human IL-2 (PeproTech) was added to the medium for 24 hours before performing assays.

Lentiviral transduction of the PD-1 engager. In a pre-coated 12-well plate, iECs were plated at a density of 5×10⁴ and then incubated overnight at 37° C. at 5% CO₂. The next day, cells were incubated overnight at 37° C., 5% CO₂ with lentiviral particles carrying a transgene for the PD-1 engager under the enhanced EF1α promoter (Gentarget, custom product). The lentiviruses further contained an RFP selection marker and were used at a multiplicity of infection of 4. Polybrene (8 μg/m, Millipore) was added to the media and the plate was centrifuged at 800 g for 30 min prior to the overnight incubation. Cell populations were sorted on FACSAria (BD Biosciences) using the RFP tag.

iEC BLINK cell killing assay. Fluc+B2M−/−CIITA−/− (DKO) iECs and B2M−/−CIITA−/− (DKO) PD-1 engager transgenic iECs were counted and plated at a concentration of 1×10³ cells per 96-well. Then all target cells were mixed with primary human NK cells at an E:T ratio of 10:1. All NK cells were preincubated with human IL-2 (Life Technologies) at a concentration of 1 g/mL for 72 h. After 4 h in the BLI killing assay, luciferase expression was detected by adding D-luciferin (Promega). As controls, target cells were left untreated or were treated with 2% Triton X-100 in cell-specific media. Signals were quantified with Ami HT (Spectral Instruments Imaging) in p/s/cm2/sr and relative BLI intensities compared to untreated target cells are measured.

Ex vivo T cell priming. Blood from an HLA-A2-negative donor was collected and PBMCs were obtained after Ficoll separation. The PBMCs were primed by co-culturing 5×10⁵ HLA-A2-positive wt iEC cells and 1×10⁶ PBMC in a gelatin-coated flasks. The media, which consisted of a 1:1 mixture of endothelial cell medium and PBMC medium, was changed every 3 days. After 14 days, the cells in suspension were harvested and sorted using an APC mouse anti-human CD3 antibody (clone SP34-2, catalog no. 557597, BD Biosciences) together with the isotype-matched control APC mouse IgG1 antibody (clone MOPC-21, catalog no. 550854, BD Biosciences), and a BV421 mouse anti-human CD8 antibody (clone SKI, catalog no. 344748, Biolegend) together with the isotype-matched control mouse IgG1 antibody (clone MOPC-21, catalog no. 400157, Biolegend). The CD3+CD8+ cells were sorted using a FACS Aria flow cytometer (BD Biosciences) and used for CD8 T cell killing assays.

iEC BLI CD8 T cell killing assay. Fluc+wt iECs and wt PD-1 engager transgenic iECs were counted and plated at a concentration of 1×10³ cells per 96-well. Then all target cells were mixed with ex vivo primed CD8 T cells at an E:T ratio of 10:1. After 4 h in the BLI killing assay, luciferase expression was detected by adding D-luciferin (Promega). As controls, target cells were left untreated or were treated with 2% Triton X-100 in cell-specific media. Signals were quantified with Ami HT (Spectral Instruments Imaging) in p/s/cm2/sr.

Human pancreatic beta cells. Human iPSC-derived pancreatic beta cells were purchased from TaKaRa (ChiPSC22, catalog no. Y10106) and were cultured in Cellartis hiPS Beta Cell Media Kit (TaKaRa, catalog no. Y10108). Cells were plated in 12-well plates according to the manufacturer protocol. Some cells were transduced with PD-1 engager lentiviral particles (GenTarget).

Islet cells (pancreatic beta cells) with and without PD-1-Engager expression were then plated on the XCelligence platform. They attached to plastic dishes but also grew in clusters and the cell index kept increasing over time. Despite the clustering growth pattern, the assay was still very sensitive in detecting target cell killing. The islet cells showed the HLA type A2. We thus used primed CD3+CD8+ T cells as effector cells, generated as described above. The unedited islet cells were killed by the primed T cells with typical T cell killing kinetics. In contrast, the islet beta cells expressing the PD-1 Engager were protected from killing by primed T cells.

To show binding of DKO PD-1 engager iECs to PD-1, we used recombinant human PD-1 protein (Fc Chimera Active, ab221398, Abcam, 0.1 ug/mL) with Mouse anti-Human IgG Fc Secondary Antibody (3D3cc, Novus Biologicals, Allophycocyanin, 1:200). Flow cytometry was done on an LSRII (BD Biosciences).

### Exemplary Sequences:

PD-1 engager with IL-2 signal peptide
SEQ ID NO: 1:
MYRMQLLSCIALSLALVINSDIQMTQSPSSLSASVGDRVTITCQASQSPNNLLAWYQQKPGK
APKLLIYGASDLPSGVPSRFSGSGSGTDFTLTISSLQPEDFATYYCQNNYYVGPVSYAFGGG
TKVEIKGGGGSGGGGSGGGGSQVQLVQSGAEVKKPGASVKVSCKVSGYSLSKYDMSWVRQAP
GKGLEWMGIIYTSGYTDYAQKFQGRVTMTEDTSTDTAYMELSSLRSEDTAVYYCATGNPYYT
NGFNSWGQGTLVTVSSTTTPAPRPPTPAPTIASQPLSLRPEACRPAAAVHTRGLDFACDAAV
LVLLVIVIISLIVLVVIW
IL-2 signal peptide
SEQ ID NO: 2:
MYRMOLLSCIALSLALVINS
CD8a hinge
SEQ ID NO: 3:
TTTPAPRPPTPAPTIASQPLSLRPEACRPAAAVHTRGLDFACD
Platelete Derived Growth Factor Receptor
(PDGFR) Transmembrane Domain (TMD)
SEQ ID NO: 4:
AAVLVLLVIVIISLIVLVVIW
CD47 Transmembrane domain (TMD)
SEQ ID NO: 5
NILIVIFPIFAILLFWGQFGIKTLKYRSGGMDEKTIALLVAGLVITVIVIVGAILFVPGEYS
LKNATGLGLIVTSTGILILLHYYVFSTAIGLTSFVIAILVIQVIAYILAVVGLSLCIAACIP
MHGPLLISGLSILALAQLLGLVYM
CD64 Transmembrane domain (TMD)
SEQ ID NO: 6:
VLFYLAVGIMFLVNTVLWVTI
CD47 Intracellular Domain (ICD)
SEQ ID NO: 7:
KFVASNQKTIQPPRKAVEEPLNAFKESKGMMNDE
Human SIRPα
>NP_001317657.1 tyrosine-protein phosphatase
non-receptor type substrate 1 isoform 2
precursor [Homo sapiens]
SEQ ID NO: 8
MEPAGPAPGRLGPLLCLLLAASCAWSGVAGEEELQVIQPDKSVLVAAGETATLRCTATSLIP
VGPIQWERGAGPGRELIYNQKEGHFPRVTTVSDLTKRNNMDFSIRIGNITPADAGTYYCVKE
RKGSPDDVEFKSGAGTELSVRAKPSAPVVSGPAARATPQHTVSFTCESHGESPRDITLKWFK
NGNELSDFQTNVDPVGESVSYSIHSTAKVVLTREDVHSQVICEVAHVTLQGDPLRGTANLSE
TIRVPPTLEVTQQPVRAENQVNVTCQVRKFYPQRLQLTWLENGNVSRTETASTVTENKDGTY
NWMSWLLVNVSAHRDDVKLTCQVEHDGQPAVSKSHDLKVSAHPKEQGSNTAAENTGSNERNI
YIVVGVVCTLLVALLMAALYLVRIRQKKAQGSTSSTRLHEPEKNAREITQVQSLDTNDITYA
DLNLPKGKKPAPQAAEPNNHTEYASIQTSPQPASEDTLTYADLDMVHLNRTPKQPAPKPEPS
FSEYASVQVPRK
Human CD47
>NP_001768.1 leukocyte surface antigen CD47
isoform 1 precursor [Homo sapiens]
SEQ ID NO: 9
MWPLVAALLLGSACCGSAQLLENKTKSVEFTFCNDTVVIPCFVTNMEAQNTTEVYVKWKFKG
RDIYTFDGALNKSTVPTDESSAKIEVSQLLKGDASLKMDKSDAVSHTGNYTCEVTELTREGE
TIIELKYRVVSWESPNENILIVIFPIFAILLFWGQFGIKTLKYRSGGMDEKTIALLVAGLVI
TVIVIVGAILFVPGEYSLKNATGLGLIVTSTGILILLHYYVFSTAIGLTSFVIAILVIQVIA
YILAVVGLSLCIAACIPMHGPLLISGLSILALAQLLGLVYMKFVASNQKTIQPPRKAVEEPL
NAFKESKGMMNDE
CD47 Extracellular Domain (ECD)
SEQ ID NO: 10
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVVSWFSPNE
CD47 Immunoglobulin Superfamily Domain
SEQ ID NO: 11:
QLLFNKTKSVEFTFCNDTVVIPCFVTNMEAQNTTEVYVKWKFKGRDIYTFDGALNKSTVPTD
FSSAKIEVSQLLKGDASLKMDKSDAVSHTGNYTCEVTELTREGETIIELKYRVV
PD-L1 (Isoform 1)
SEQ ID NO: 12:
MRIFAVFIFMTYWHLLNAFTVTVPKDLYVVEYGSNMTIECKFPVEKQLDLAALIVYWEMEDK
NIIQFVHGEEDLKVQHSSYRQRARLLKDQLSLGNAALQITDVKLQDAGVYRCMISYGGADYK
RITVKVNAPYNKINQRILVVDPVTSEHELTCQAEGYPKAEVIWTSSDHQVLSGKTTTTNSKR
EEKLENVTSTLRINTTTNEIFYCTFRRLDPEENHTAELVIPELPLAHPPNERTHLVILGAIL
LCLGVALTFIFRLRKGRMMDVKKCGIQDTNSKKQSDTHLEET
PD-L1 (Isoform 2)
SEQ ID NO: 13:
MRIFAVFIFMTYWHLLNAPYNKINQRILVVDPVTSEHELTCQAEGYPKAEVIWTSSDHQVLS
GKTTTTNSKREEKLENVTSTLRINTTTNEIFYCTFRRLDPEENHTAELVIPELPLAHPPNER
THLVILGAILLCLGVALTFIFRLRKGRMMDVKKCGIQDTNSKKQSDTHLEET
PD-L1 (Isoform 3)
SEQ ID NO: 14:
MRIFAVFIFMTYWHLLNAFTVTVPKDLYVVEYGSNMTIECKFPVEKQLDLAASIVYWEMEDK
NIIQFVHGEEDLKVQHSSYRQRARLLKDQLSLGNAALQITDVKLQDAGVYRCMISYGGADYK
RITVKVNAPYNKINQRILVVDPVTSEHELTCQAEGYPKAEVIWTSSDHQVLSGD
PD-L2 (Isoform 1)
SEQ ID NO: 15:
MIFLLLMLSLELQLHQIAALFTVTVPKELYIIEHGSNVTLECNEDTGSHVNLGAITASLQKV
ENDTSPHRERATLLEEQLPLGKASFHIPQVQVRDEGQYQCIIIYGVAWDYKYLTLKVKASYR
KINTHILKVPETDEVELTCQATGYPLAEVSWPNVSVPANTSHSRTPEGLYQVTSVLRLKPPP
GRNFSCVFWNTHVRELTLASIDLQSQMEPRTHPTWLLHIFIPFCIIAFIFIATVIALRKQSC
QKLYSSKDTTKRPVTTTKREVNSAI
PD-L2 (Isoform 2)
SEQ ID NO: 16:
MIFLLLMLSLELQLHQIAALFTVTVPKELYIIEHGSNVTLECNFDTGSHVNLGAITASLQKV
ENDTSPHRERATLLEEQLPLGKASFHIPQVQVRDEGQYQCIIIYGVAWDYKYLTLKVKGQME
PRTHPTWLLHIFIPFCIIAFIFIATVIALRKQLCQKLYSSKDTTKRPVTTTKREVNSAI
PD-L2 (Isoform 3)
SEQ ID NO: 17:
MIFLLLMLSLELQLHQIAALFTVTVPKELYIIEHGSNVTLECNEDTGSHVNLGAITASLQKV
ENDTSPHRERATLLEEQLPLGKASFHIPQVQVRDEGQYQCIIIYGVAWDYKYLTLKVKDGTQ
DPSNLAASHFHPLLHHCFHFHSHSDSPKKTTLSKAVFFKRHNKKTCHHNKEGSEQCYL
Antibody 1 Heavy Chain CDR1
SEQ ID NO: 18:
KVSGYSLSKYDMS
Antibody 1 Heavy Chain CDR2
SEQ ID NO: 19:
IIYTSGYTDYAQKFQG
Antibody 1 Heavy Chain CDR3
SEQ ID NO: 20:
ATGNPYYTNGENS
Antibody 1 Light Chain CDR1
SEQ ID NO: 21:
QASQSPNNLLA
Antibody 1 Light Chain CDR2
SEQ ID NO: 22:
YGASDLPS
Antibody 1 Light Chain CDR3
SEQ ID NO: 23:
QNNYYVGPVSYA
PD-1 engager with CD8a signal peptide
and myc-tag
SEQ ID NO: 24:
MALPVTALLLPLALLLHAARPEQKLISEEDLDIQMTQSPSSLSASVGDRVTITCQASQSPNN
LLAWYQQKPGKAPKLLIYGASDLPSGVPSRFSGSGSGTDFTLTISSLQPEDFATYYCQNNYY
VGPVSYAFGGGTKVEIKGGGGSGGGGSGGGGSQVQLVQSGAEVKKPGASVKVSCKVSGYSLS
KYDMSWVRQAPGKGLEWMGIIYTSGYTDYAQKFQGRVTMTEDTSTDTAYMELSSLRSEDTAV
YYCATGNPYYTNGFNSWGQGTLVTVSSTTTPAPRPPTPAPTIASQPLSLRPEACRPAAAVHT
RGLDFACDAAVLVLLVIVIISLIVLVVIW
CD8a signal peptide
SEQ ID NO: 25:
MALPVTALLLPLALLLHAARP
myc-tag
SEQ ID NO: 26:
EQKLISEEDL
TIM3 Engager Protein
SEQ ID NO: 27:
MALPVTALLLPLALLLHAARPEQKLISEEDLQMQLVQSGGEVKKPGASVKVSCKTSGYRFTS
YGISWVRQAPGQGLEWMGWISGYNGETNYAETLQGRLTLTTDTSTSTAYMELGSLRPDDTAV
YYCTRDGHSPYFDYWGQGTLVTVSSGGGGSGGGGSGGGGSQAVLTQPASVSGSPGQSVTISC
TGTSSDVGGYNYVSWYQQHPGKAPKLMIYEVSKRPSGIPERFSGSNSGNTATLTISRVEAGD
EADYYCQVWDSSSDHWVFGRGTKLTVLTTTPAPRPPTPAPTIASQPLSLRPEACRPAAAVHT
RGLDFACDAAVLVLLVIVIISLIVLVVIW
CD8a Signal Peptide
SEQ ID NO: 28:
MALPVTALLLPLALLLHAARP
Heavy Chain Variable Region (V_{H})
SEQ ID NO: 29:
QMQLVQSGGEVKKPGASVKVSCKTSGYRFTSYGISWVRQAPGQGLEWMGWISGYNGETNYAE
TLQGRLTLTTDTSTSTAYMELGSLRPDDTAVYYCTRDGHSPYFDYWGQGTLVTVSS
(GGGG)₃ Linker
SEQ ID NO: 30:
GGGGSGGGGSGGGGS
Light Chain Variable Region (V_{L})
SEQ ID NO: 31:
QAVLTQPASVSGSPGQSVTISCTGTSSDVGGYNYVSWYQQHPGKAPKLMIYEVSKRPSGIPE
RFSGSNSGNTATLTISRVEAGDEADYYCQVWDSSSDHWVFGRGTKLTVL
LILRB3 Engager Protein
SEQ ID NO: 32:
MALPVTALLLPLALLLHAARPEQKLISEEDLEVQLLESGGGLVQPGGSLRLSCAASGFTESS
YAMSWVRQAPGKGLEWVSAISGSGGSTYYADSVKGRFTISRDNSKNTLYLOMNSLRAEDTAV
YYCARRKKRERGFSGNDPVGAIDVWGQGTLVTVSSGGGGSGGGGSGGGGSQSVLTQPPSASG
TPGQRVTISCTGSSSNIGAGYDVHWYQQLPGTAPKLLIYGNTNRPSGVPDRESGSKSGTSAS
LAISGLRSEDEADYYCSAWDDSLSGVVFGGGTKLTVLGTTTPAPRPPTPAPTIASQPLSLRP
EACRPAAAVHTRGLDFACDAAVLVLLVIVIISLIVLVVIW
Heavey Chain Variable Region (V_{H})
SEQ ID NO: 33:
EVQLLESGGGLVQPGGSLRLSCAASGFTESSYAMSWVRQAPGKGLEWVSAISGSGGSTYYAD
SVKGRFTISRDNSKNTLYLQMNSLRAEDTAVYYCARRKKRERGFSGNDPVGAIDVWGQGTLV
TVSS
Light Chain Variable Region (V_{L})
SEQ ID NO: 34:
QSVLTQPPSASGTPGQRVTISCTGSSSNIGAGYDVHWYQQLPGTAPKLLIYGNTNRPSGVPD
RFSGSKSGTSASLAISGLRSEDEADYYCSAWDDSLSGVVFGGGTKLTVLG
LILRB1 Engager Protein
SEQ ID NO: 35:
MALPVTALLLPLALLLHAARPEQKLISEEDLEQSLEESGGGLVKPGASLTVTCAVSGESLNS
YAITWVRQAPGKGLEYIGYIDTGASITDYASWAKGRFTISKTSSTTVTLEMTSLTDADTATY
FCARGESMFKLWGPGTLVTISSGGGGSGGGGSGGGGSELDLTQTPSSVSAAVGGTVTISCQA
SQSVYGNNRLAWYHQKPGQPPKRLIYLASTLDSGVPSRFKGAGSGTQFTLTISDLECDDAAT
YYCAGGYAGNFNAFGGGTEVEIKTTTPAPRPPTPAPTIASQPLSLRPEACRPAAAVHTRGLD
FACDAAVLVLLVIVIISLIVLVVIW
Heavey Chain Variable Region (V_{H})
SEQ ID NO: 36:
EQSLEESGGGLVKPGASLTVTCAVSGESLNSYAITWVRQAPGKGLEYIGYIDTGASITDYAS
WAKGRFTISKTSSTTVTLEMTSLTDADTATYFCARGESMEKLWGPGTLVTISS
Light Chain Variable Region (V_{L})
SEQ ID NO: 37:
ELDLTQTPSSVSAAVGGTVTISCQASQSVYGNNRLAWYHQKPGQPPKRLIYLASTLDSGVPS
RFKGAGSGTQFTLTISDLECDDAATYYCAGGYAGNENAFGGGTEVEIK

All publications and patent documents disclosed or referred to herein are incorporated by reference in their entirely. The foregoing description has been presented only for purposes of illustration and description. This description is not intended to limit the invention to the precise form disclosed. It is intended that the scope of the invention be defined by the claims appended hereto.

What is claimed:

1. An Immune Checkpoint Engager (ICE) cell, comprising an engager molecule expressed on a cell surface, wherein said engager molecule engages with an immune checkpoint molecule on an immune cell, wherein said engager molecule is expressed at least at a level that protects said ICE cell from being killed by said immune cell.

2. The ICE cell of claim 1, wherein said engager molecule is a protein that does not engage SIRPα on said immune cell.

3. The ICE cell of claim 2, wherein said engager molecule engages PD-1.

4. The ICE cell of claim 2, wherein said protein comprises an immunoglobulin domain that binds PD-1.

5. The ICE cell of claim 4, wherein said immunoglobulin domain is an antibody VH domain.

6. The ICE cell of claim 5, wherein said antibody VH domain comprises at least a 90% sequence identity to amino acid numbers 146-264 of SEQ ID NO:1.

7. The ICE cell of claim 6, wherein said antibody VH domain comprises the sequence of amino acid numbers 146-264 of SEQ ID NO:1.

8. The ICE cell of claim 4, wherein said immunoglobulin domain is an antibody VL domain.

9. The ICE cell of claim 8, wherein said antibody VL domain comprises at least a 90% sequence identity to amino acid numbers 21-130 of SEQ ID NO:1.

10. The ICE cell of claim 9, wherein said antibody VH domain comprises the sequence of amino acid numbers 21-130 of SEQ ID NO:1.

11. The ICE cell of any one of claims 2-10, wherein said protein is a fusion protein.

12. The ICE cell of claim 11, wherein said fusion protein comprises at least a 90% sequence identity to the IL-2 signal peptide of SEQ ID NO:2.

13. The ICE cell of claim 12, wherein said fusion protein comprises the IL-2 signal peptide of SEQ ID NO:2.

14. The ICE cell of claim 11, wherein said fusion protein comprises at least a 90% sequence identity to the CD8a hinge peptide of SEQ ID NO:3.

15. The ICE cell of claim 14, wherein said fusion protein comprises the CD8a hinge peptide of SEQ ID NO:3.

16. The ICE cell of claim 11, wherein said fusion protein comprises at least a 90% sequence identity to the PDGFR transmembrane domain (TMD) of SEQ ID NO:4.

17. The ICE cell of claim 16, wherein said fusion protein comprises the PDGFR TMD of SEQ ID NO:4.

18. The ICE cell of claim 11, wherein said engager molecule is a protein having at least a 90% sequence identity to SEQ ID NO:1.

19. The ICE cell of claim 18, wherein said engager molecule has the sequence identity of SEQ ID NO:1.

20. The ICE cell of any one of claims 1-11, wherein said engager molecule comprises an antibody Fab or a single chain variable fragment (scFV) that binds to PD-1.

21. The ICE cell of claim 20, wherein said Fab or scFV binds to PD-1 with an affinity measured by its dissociation constant (Kd), wherein said Kd is between about 10⁻⁷ and 10⁻¹³ M.

22. The ICE cell of any one of claims 1-11, wherein said engager molecule comprises one or more antibody complementarity determining regions (CDRs) that binds to PD-1.

23. The ICE cell of claim 22, wherein said one or more CDRs have at least a 90% sequence identity to any one of SEQ ID NOS:18-23.

24. The PD1 engager cell of any one of claims 1-15, wherein said engager molecule is a fusion protein comprising a heterologous transmembrane domain (TMD).

25. The ICE cell of claim 24, wherein said TMD comprises a single a helix, multiple a helices, or a rolled-up β sheet.

26. The ICE cell of claim 24, wherein said heterologous TMD is selected from the group consisting of CD85f, CD349, CD284, CD261, CD172b, CD277, CD186, CD156c, CD304, CD254, CD263, CD267, CD337, CD170, CD283, CD133, CD327, CD205, CD232, CD282, CD16b, CD85i, CD85a, CD85c, CD275, CD108, CD358, CD335, CD218b, CD355, CD336, CD160, CD25, CD4, CD8a, CD235a, CD233, CD230, CD90, CD74, CD3d, CD340, CD236, CD61, CD18, CD54, CD29, CD1a, CD5, CD220, CD2, CD66e, CD51, CD141, CD115, CD42b, CD221, CD271, CD55, CD243, CD98, CD10, CD41, CD14, CD45, CD228, CD16a, CD49e, CD126, CD63, CD48, CD7, CD140b, CD3g, CD117, CD28, CD8b, CD37, CD11b, CD107a, CD331, CD222, CD20, CD79a, CD32, CD143, CD324, CD42c, CD107b, CD56, CD102, CD49d, CD66a, CD142, CD59, CD62L, CD121a, CD122, CD13, CD155, CD119, CD19, CD116, CD46, CD1e, CD1d, CD227, CD44, CD62P, CD104, CD43, CD140a, CD31, CD152, CD326, CD62E, CD36, CD127, CD49b, CD105, CD35, CD223, CD138, CD325, CD58, CD106, CD53, CD120a, CD224, CD21, CD33, CD22, CD120b, CD11a, CD11c, CD363, CD73, CD88, CD204, CD332, CD9, CD203a, CD334, CD333, CD206, CD49f, CD238, CD252, CD89, CD124, CD181, CD182, CD24, CD95, CD40, CD49c, CD159a, CD159c, CD314, CD27, CD123, CD26, CD82, CD121b, CD34, CD38, CD30, CD1b, CD1c, CD154, CD6, CD52, CD132, CD32, CD66b, CD171, CD191, CD197, CD185, CD131, CD50, CD70, CD153, CD144, CD80, CD362, CD68, CD361, CD147, CD309, CD135, CD292, CD103, CD130, CD42d, CD66d, CD66c, CD96, CD110, CD79b, CD200, CD192, CD231, CD86, CD212, CD118, CD146, CD134, CD158a, CD158b1, CD158b2, CD158e, CD158k, CD158j, CD158i, CD178, CD295, CD151, CD97, CD183, CD39, CD239, CD193, CD194, CD195, CD196, CDw198, CDw199, CD296, CD298, CD49a, CD322, CD85g, CD184, CD172a, CD156a, CD339, CD156b, CD213a1, CD129, CD83, CD125, CD241, CD269, CD202b, CD87, CD164, CD136, CD137, CD249, CD69, CD91, CDw210b, CD167a, CD300c, CD157, CD317, CD148, CD161, CD215, CD150, CD11d, CD218a, CD210, CD166, CD162, CD213a2, CD242, CD158g, CD158h, CD279, CD111, CD281, CD226, CD234, CD167b, CD300e, CD276, CD305, CD300g, CD300d, CD109, CD272, CD163, CD302, CD158f1, CD85h, CD85d, CD177, CD158z, CD158f2, CD85j, CD300f, CD92, CD351, CD112, CD100, CD270, CD101, CD297, CD316, CD352, CD217, CD307b, CD307a, CD307c, CD307d, CD307e, CD114, CD180, CD158d, CD273, CD290, CD244, CD169, CD299, CD318, CD360, CD229, CD248, CD354, CD320, CD93, CD319, CD113, CD163b, CD289, CD288, CD329, CD274, CD353, CD172g, CD315, CD280, CD264, CD300a, CD312, CD84, CD344, CD350, CD246, CD201, CD338, CD208, CD257, CD328, CD286, CD357, CD294, CD321, CD265, CD278, ITGA7, ITGA8, ITGA9, ITGA10, ITGA11, CD51, CD41, CD29, CD18, CD61, CD104, CD47 (SEQ ID NO:5), CD64 (SEQ ID NO:6), and PDGFR (SEQ ID NO:4).

27. The ICE cell of claim 24, wherein said TMD comprises a sequence with at least a 90% sequence identity to SEQ ID NO:5 or SEQ ID NO:6.

28. The ICE cell of claim 24, wherein said TMD comprises the sequence of SEQ ID NO:5 or SEQ ID NO:6.

29. The ICE cell of claim 24, wherein said TMD is from a 7 transmembrane protein (7TM) or an immunoglobulin cell-surface protein.

30. The ICE cell of any one of claims 1-29, wherein said engager molecule does not have an intracellular domain (ICD).

31. The ICE cell of any one of claims 1-29, wherein said engager molecule has an intracellular domain from CD16, CD32, CD64, CD8, CD3, CD28, or CD137.

32. The ICE cell of any one of claim 2 or 4-31, wherein said engager molecule has one or more linker or hinge regions connecting ECD, TMD, or ICD sequences.

33. The ICE cell of any one of claim 2 or 4-32, wherein said protein is an antibody, receptor, ligand, or adhesion protein.

34. The ICE cell of any one of claims 1-33, further comprising a reduced or eliminated HLA-I or HLA-II expression.

35. The ICE cell of any one of claims 1-34, wherein said cell is ABO blood group type O.

36. The ICE cell of any one of claims 1-35, wherein said cell is Rhesus factor negative (Rh−).

37. The ICE cell of any one of claims 1-36, wherein said cell has a reduced or eliminated ABO blood group antigen selected from the group consisting of A1, A2, and B.

38. The ICE cell of any one of claims 1-37, wherein said cell has a reduced or eliminated Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S.

39. The ICE cell of any one of claims 1-38, wherein the cell is a hypoimmunogenic (HI) cell comprising: an endogenous Major Histocompatibility Complex Class I (HLA-I) function that is reduced when compared to an unmodified parental cell and an endogenous Major Histocompatibility Complex Class II (HLA-II) function that is reduced when compared to said unmodified parental cell.

40. The ICE cell of any one of claims 1-39, wherein said engager cell comprises modulated expression of one or more of HLA-I human leukocyte antigens, HLA-II human leukocyte antigens, CD64, CD47, CD38, CCR5, CXCR4, NLRC5, CIITA, B2M, HLA-A, HLA-B, HLA-C, HLA-E, HLA-G, PD-L1, CTLA-4-Ig, CD47, CI-inhibitor, IL-35, RFX-5, RFXAP, RFXANK, NFY-A, NFY-B, NFY-C, IRF-1, OX40, GITR, 4-1BB, CD28, B7-1, B7-2, ICOS, CD27, HVEM, SLAM, CD226, PD1, CTL4, LAG3, TIGIT, TIM3, CD160, BTLA, CD244, CD30, TLT, VISTA, B7-H3, PD-L2, LFA-1, CD2, CD58, ICAM-3, TCRA, TCRB, FOXP3, HELIOS, ST2, PCSK9, APOC3, CD200, FASLG, CLC21, MFGE8, SERPIN B9, TGFβ, CD73, CD39, LAG3, IL1R2, ACKR2, TNFRSF22, TNFRSF23, TNFRS10, DAD1, PVR, or IFNγR1 d39 relative to a parental cell, wherein said engager cell is ABO blood group type O or Rhesus factor negative (Rh−).

41. The ICE cell of any one of claims 1-40, further comprising an elevated expression of an antibody Fc receptor on the cell surface, wherein said Fc receptor helps to evade antibody dependent cellular cytotoxicity (ADCC) or complement mediated cytotoxicity (CDC).

42. The ICE cell of claim 41, wherein said Fc receptor is CD16, CD32, CD64, or truncated CD64.

43. The ICE cell of any one of claims 1-42, wherein said cell is pluripotent.

44. The ICE cell of claim 43, wherein said cell is a hypoimmune pluripotent (HIP) cell.

45. The ICE cell of claim 44, wherein said cell is a hypoimmune pluripotent cell having an ABO blood type O (HIPO).

46. The ICE cell of claim 45, wherein said cell is a hypoimmune pluripotent cell is Rh factor negative (HIP−).

47. The ICE cell of claim 46, wherein said cell is a hypoimmune pluripotent cell having an ABO blood type O and is Rh factor negative (HIPO−).

48. The ICE cell of of any one of claims 43-47, wherein said cell is a pluripotent (PSC) cell, induced PSC (iPSC), or an embryonic stem cell (ESC).

49. The ICE cell of any one of claims 1-42, wherein said engager cell is a specific tissue type.

50. The ICE cell of claim 49 wherein said cell is a chimeric antigen receptor (CAR) cell, a T cell, an NK cell, an endothelial cell, a dopaminergic neuron, a cardiac cell, a pancreatic islet beta cell, thyroid epithelial cells, parathyroid cells, or a retinal pigment epithelium cell.

51. The ICE cell of claim 49, wherein said cell is a CAR-T cell, a CAR-NK cell, a TCR T cell, or a TCR NK cell.

52. The ICE cell of any one of claims 1-42 or 49-51, wherein said engager cell is differentiated from a pluripotent cell.

53. A pharmaceutical composition, comprising the ICE cell of any one of claims 49-52 and a pharmaceutically-acceptable carrier.

54. A medicament, comprising the ICE cell of any one of claims 49-52 and a pharmaceutically-acceptable carrier.

55. A method of treating a disease in a subject, comprising transplanting the ICE cell of any one of claims 49-52 into said subject.

56. The method of claim 55, wherein said disease is Type 1 diabetes, a cardiac disease, a neurological disease, an endocrine disease, cancer, blindness, or a vascular disease.

57. A use of the ICE cell of any one of claims 49-52 for preparing a pharmaceutical composition for treating a disease in a subject.

58. A use of The ICE cell of any one of claims 49-52 for treating a disease in a subject.

59. The use of either one of claim 57 or 58, wherein said disease is Type 1 diabetes, a cardiac disease, a neurological disease, an endocrine disease, cancer, blindness, or a vascular disease.

60. The ICE cell of any one of claims 1-52, further comprising a Signal Regulatory Protein Alpha (SIRPα) engager molecule on said cell surface that engages with a SIRPα protein on said immune cell, wherein said SIRP-α engagement prevents said engager cell from being killed by said immune cell, wherein said SIRP-α engager cell surface molecule lacks a functional intracellular domain.

61. The ICE cell of claim 60, wherein said engager molecule is a protein.

62. The ICE cell of claim 61, wherein said protein is a fusion protein.

63. The ICE cell of claim 62, wherein said fusion protein comprises a CD47 extracellular domain (ECD).

64. The ICE cell of any one of claims 60-63, wherein said engager molecule comprises an immunoglobulin superfamily domain.

65. The ICE cell of any one of claims 60-64, wherein said SIRPα engager molecule comprises one or more antibody complementarity determining regions (CDRs) that binds to SIRPα.

66. The ICE cell of claim 65, wherein said CDRs comprise a sequence having at least a 90% sequence identity to any one of SEQ ID NOS:18-23.

67. The ICE cell of claim 66, wherein said CDRs comprise any one of the sequences of SEQ ID NOS:18-23.

68. The ICE cell of claim 2, wherein said engager molecule engages with one or more immune cell receptors selected from the group consisting of PD-1, TIM3, LILRB3, and LILRB1.

69. The ICE cell of any one of claims 1-53 or 68, further comprising a safety switch.

70. The ICE cell of claim 69, wherein said safety switch is a suicide gene.

71. The ICE cell of claim 70, wherein said suicide gene is (a) a herpes simplex virus thymidine kinase gene (HSV-tk) that has a ganciclovir trigger, (b) an Escherichia coli cytosine deaminase gene (EC-CD) that has a 5-fluorocytosine (5-FC) trigger, or (c) an inducible iCasp9 protein that has an AP1903 trigger.
